(19) United States
(12) Patent Application Publication
(10) Pub. No.: US 2012/0203076 A1
(43) Pub. Date: Aug. 9, 2012

(54) Portable Physiological Data Monitoring Device

(76) Inventors: Jean Pierre Fatta, Naples, FL (US); Michael Sean Hynson, Ft. Myers, FL (US); Curtis Wayne Boyatt, Naples, FL (US); Richard JohnJuda, Estero, FL (US); Mary Christine DeGroot, Estero, FL (US)

(21) Appl. No.: 13/023,521

(22) Filed: Feb. 8, 2011

(51) Int. Cl. A61B 5/00

(52) U.S. Cl. 600/300

(57) ABSTRACT

A portable monitoring device and a method for monitoring and alerting physiological parameters of a patient are provided. The portable monitoring device comprises a housing, parameter sensing devices, a processing unit, and data communication units. The housing defines an inner surface for establishing physical contact with the patient's body part, and an outer surface opposing the inner surface. The parameter sensing devices acquire physiological data associated with the patient's physiological parameters. The processing unit processes the acquired physiological data and patient information. The data communication units, capable of synchronizing with responder devices, transmit the processed physiological data and the patient information to a base monitoring unit, a remote monitoring station, and/or responder devices via one or more transceivers and data communication interfaces for initiating relief measures. The portable monitoring device monitors the patient's physiological parameters and alerts the remote monitoring station for providing the relief measures to the patient.

### BACKGROUND

The portable monitoring device and system disclosed herein, in general, relates to monitoring vital parameters of a patient. More particularly, the portable monitoring device and system disclosed herein relates to monitoring vital parameters of the patient and for alerting a remote monitoring station for dispatching and providing immediate relief to the patient.

Systems that monitor vital parameters of the patient from a remote station often fail to locate the patient in emergencies to deploy a relief team to the patient. Moreover, these systems that monitor vital parameters of patients of an elderly age group, for example, adult patients may not be suitable for patients belonging to other age groups, for example, infants. Conventional systems for monitoring vital parameters of the patient from a remote station often provide expensive solutions to the patients.

Another unaddressed market requiring attention is the immediate detection of sudden infant death syndrome (SIDS). There is a need for a device and system that addresses problems related to the immediate detection and notification of SIDS in infants. Moreover, there is a need for a monitoring device that detects conditions, for example, fever, temperature, heart rate, etc., in infants for monitoring the general health of the infants.

There is a need for a method and system that monitors the general well being of a patient of every age group and spectrum of health. Furthermore, the technologies used for designing the method and system need to be clearly defined and acknowledged by medical professionals. The technologies need to be priced to appeal to the managed-care market, which stresses the importance of low cost of total treatment parameters, as well as to parents of infant children in the age group of 0-12 months.

There is also a need for an immediate response system when the vitals of the patient exceed set thresholds. The quicker the response, the more likely is the chance of treatment and recovery of the patient.

Hence, there is a long felt but unresolved need for a cost-effective portable monitoring device and system that monitors vital parameters of a patient, for example, an adult patient, an infant, etc., and patients who require round the clock monitoring. Furthermore, there is a need for a portable monitoring device that immediately locates the patient in emergencies and deploys medical practitioners to the patient.

### SUMMARY OF THE INVENTION

This summary is provided to introduce a selection of concepts in a simplified form that are further described in the detailed description of the invention. This summary is not intended to identify key or essential inventive concepts of the claimed subject matter, nor is it intended for determining the scope of the claimed subject matter.

The portable monitoring device, system, and method disclosed herein address the above mentioned need for a health monitoring device system that monitors vital parameters of a patient while providing a cost effective solution for emergency medical services.

A portable monitoring device, system, and method for monitoring physiological parameters of a patient and alerting a remote monitoring station based on the monitored physiological parameters are provided. The portable monitoring device comprises a housing, one or more parameter sensing devices, a processing unit, one or more transceivers, and one or more data communication units. The housing is configured to conform to a body part of a patient, for example, to the wrist of a patient. In an embodiment, the housing is configured as a sleeve that can be worn on the patient's arm. The housing defines an inner surface for establishing physical contact with the patient's body part, and an outer surface opposing the inner surface. The housing comprises a fastening and resizing assembly comprising one or more extensible sliding members for resizing the portable monitoring device to conform to the patient's body part and/or for repositioning the parameter sensing devices on the housing to pick up optimum readings associated with the physiological parameters of the patient. The fastening and resizing assembly further comprises an insert and a multi-position clasp. The multi-position clasp receives the insert and secures the portable monitoring device to the patient's body part.

One or more parameter sensing devices are located at predetermined positions on the inner surface of the housing for acquiring physiological data associated with the physiological parameters of the patient by establishing physical contact with the patient's body part. The physiological parameters of the patient comprise, for example, heartbeat, an electrocardiograph (ECG), temperature, perspiration, etc., of the patient. The portable monitoring device further comprises a storage unit disposed within the housing for storing the acquired physiological data and patient information of the patient, and for providing backup of the acquired physiological data and the patient information. The patient information comprises, for example, health information of the patient, billing information of the patient, insurance information of the patient, historical patient information, baseline values for the physiological parameters of the patient, and any combination thereof.

The processing unit is disposed within the housing and is in operative communication with the parameter sensing devices for processing the acquired physiological data and the patient information. For example, the processing unit processes heart rate output signals, ECG output signals, etc., acquired from the parameter sensing devices and converts the acquired output signals, for example, from analog signals to digital signals to obtain the heart rate, ECG readings, etc. The portable monitoring device further comprises one or more location determination units, for example, a geographical positioning system (GPS) unit disposed within the housing. The location determination units are in communication with the processing unit for determining geographic location of the patient. The portable monitoring device further comprises an alerting unit in communication with the processing unit for remotely alerting a remote monitoring station for dispatching immediate responders and/or for locally alerting persons around the patient, for example, via an audio mode, a visual mode, and any combination thereof, when the acquired physiological data associated with the physiological parameters of the patient exceeds baseline parameter values.

One or more data communication units are disposed within the housing of the portable monitoring device. The data communication units are in communication with the processing unit for transmitting the processed physiological data and the patient information to one or more of a base monitoring unit, the remote monitoring station, and external responder devices via one or more transceivers and data communication interfaces disposed within the housing. The transceivers transmit the processed physiological data and the patient information, for example, in a wired mode of communication, a wireless mode of communication, or a combination thereof. The processed physiological data and the patient information transmitted to the remote monitoring station are updated a medical history log of the patient in the remote monitoring station.

The data communication units, for example, devices enabled with Bluetooth, radio frequency identification (RFID), WiFi, etc., are capable of synchronizing with the external responder devices for instant access to the physiological data of the patient and the patient information for initiating relief measures. The data communication units, for example, RFID enabled devices are also used for close proximity triangulation of the portable monitoring device in the absence of a GPS unit or in conjunction with the GPS unit on the portable monitoring device. The portable monitoring device therefore monitors the physiological parameters of the patient and alerts the remote monitoring station for providing the relief measures to the patient.

The portable monitoring device disclosed herein further comprises one or more user interface elements located at predetermined positions on the housing for providing an audiovisual indication of, for example, functioning of the portable monitoring device, power levels of the portable monitoring device, disconnection of the parameter sensing devices, operating status of the portable monitoring device, etc. The portable monitoring device further comprises a unique identifier provided on the inner surface and/or the outer surface of the portable monitoring device for identifying the patient associated with the portable monitoring device and/or for logging the acquired physiological data and the patient information into a medical account of the patient for retrieval of the acquired physiological data and the patient information from the remote monitoring station.

The portable monitoring device further comprises emergency contact information provided on the inner surface and/or the outer surface of the housing of the portable monitoring device for enabling provision of relief measures to the patient. The portable monitoring device further comprises one or more battery units located inside the housing of the portable monitoring device for powering the portable monitoring device.

The system disclosed herein comprises a base monitoring unit generally disposed in the vicinity of the portable monitoring device for charging the portable monitoring device, storing the acquired physiological data of the patient and the patient information, and establishing communication with the remote monitoring station via one or more transceivers and data communication interfaces of the base monitoring unit. The base monitoring unit is positioned, for example, in a convenient location in the vicinity of the portable monitoring device.

The system disclosed herein further comprises a remote monitoring station in communication with the portable monitoring device and the base monitoring unit via a network. The remote monitoring station receives the processed physiological data and the patient information from the portable monitoring device. In an embodiment, the remote monitoring station stores the received physiological data and the patient information in a cloud-computing environment for universal access and transmission. The portable monitoring device is configured to facilitate the transfer of medical information across the world securely, for example, via a satellite or the cloud-computing environment. The remote monitoring station updates the received physiological data and the patient information in a medical history database. The system disclosed herein further comprises one or more external responder devices capable of synchronizing with the portable monitoring device and the base monitoring unit and establishing communication with the remote monitoring station. The external responder devices synchronize with the data communication units in the portable monitoring device via the transceivers and data communication interfaces of the portable monitoring device to retrieve the processed physiological data and the patient information from the portable monitoring device.

In an embodiment, the portable monitoring device is configured as a sleeve garment for housing multiple data communication interfaces for connecting multiple peripheral devices, for example, pace makers, automated implantable cardioverter-defibrillator (AICD), intracranial devices, nasal units with mouthpieces, etc., to the portable monitoring device.

### BRIEF DESCRIPTION OF THE DRAWINGS

The foregoing summary, as well as the following detailed description of the invention, is better understood when read in conjunction with the appended drawings. For the purpose of illustrating the invention, exemplary constructions of the invention are shown in the drawings. However, the invention is not limited to the specific methods and instrumentalities disclosed herein.

FIG. 1A exemplarily illustrates a perspective view of a portable monitoring device.

FIG. 1B exemplarily illustrates a perspective view of the portable monitoring device, showing a fastening and resizing assembly of the portable monitoring device.

FIGS. 2A-2D exemplarily illustrate orthogonal views of the portable monitoring device.

FIG. 3A exemplarily illustrates a bottom orthogonal view of the portable monitoring device, showing an inner surface defined by a housing of the portable monitoring device.

FIG. 3B exemplarily illustrates a top orthogonal view of the portable monitoring device, showing an outer surface defined by the housing of the portable monitoring device.

FIG. 4 exemplarily illustrates an internal perspective view of the portable monitoring device, showing internal components of the portable monitoring device.

FIG. 5 exemplarily illustrates an orthogonal view of a parameter sensing device of the portable monitoring device.

FIG. 6 exemplarily illustrates a fastening and resizing assembly of the portable monitoring device for fastening and resizing the portable monitoring device on a body part of a patient.

FIGS. 7A-7B exemplarily illustrate a perspective view of the portable monitoring device, showing a detachable section housing a liquid crystal display that provides visual indications to the patient.

FIG. 8 exemplarily illustrates a perspective view of an embodiment of the portable monitoring device, showing multiple battery units that provide additional power supply to the portable monitoring device.

FIGS. 9A-9E exemplarily illustrate multiple views of an embodiment of the portable monitoring device.

FIG. 10 exemplarily illustrates an internal perspective view of an embodiment of the portable monitoring device, showing internal components of the portable monitoring device.

FIGS. 11A-11B exemplarily illustrate a perspective view of an embodiment of the portable monitoring device, showing a detachable section that houses light emitting diodes for indicating operating status of the portable monitoring device.

FIG. 11C exemplarily illustrates a perspective view of the portable monitoring device, showing the detachable section of FIG. 11B attached to the housing of the portable monitoring device.

FIG. 12A exemplarily illustrates a side orthogonal view of an embodiment of the portable monitoring device configured as an open-ended band.

FIG. 12B exemplarily illustrates a top orthogonal view of an embodiment of the portable monitoring device, showing internal components of the portable monitoring device.

FIGS. 13A-13C exemplarily illustrate a portable monitoring device configured to be placed on an infant's hand to monitor the infant.

FIGS. 14A-14B exemplarily illustrate orthogonal views showing a data communication unit and a data communication interface of the portable monitoring device.

FIG. 15 exemplarily illustrates a portable monitoring device configured as a sleeve garment for housing data communication interfaces that interface peripheral devices to the portable monitoring device.

FIG. 16 exemplarily illustrates an application of the portable monitoring device for child care.

FIG. 17A exemplarily illustrates a front orthogonal view of a base monitoring unit.

FIG. 17B exemplarily illustrates a rear orthogonal view of the base monitoring unit.

FIG. 17C exemplarily illustrates a top orthogonal view of the base monitoring unit.

FIG. 17D exemplarily illustrates a bottom orthogonal view of the base monitoring unit.

FIG. 18 illustrates a system for monitoring and alerting physiological parameters of a patient using the portable monitoring device.

FIG. 19 illustrates a method for monitoring and alerting physiological parameters of a patient using the portable monitoring device.

FIG. 20 exemplarily illustrates multiple peripheral devices capable of connecting to the portable monitoring device via data communication interfaces for monitoring and alerting physiological parameters of a patient.

### DETAILED DESCRIPTION OF THE INVENTION

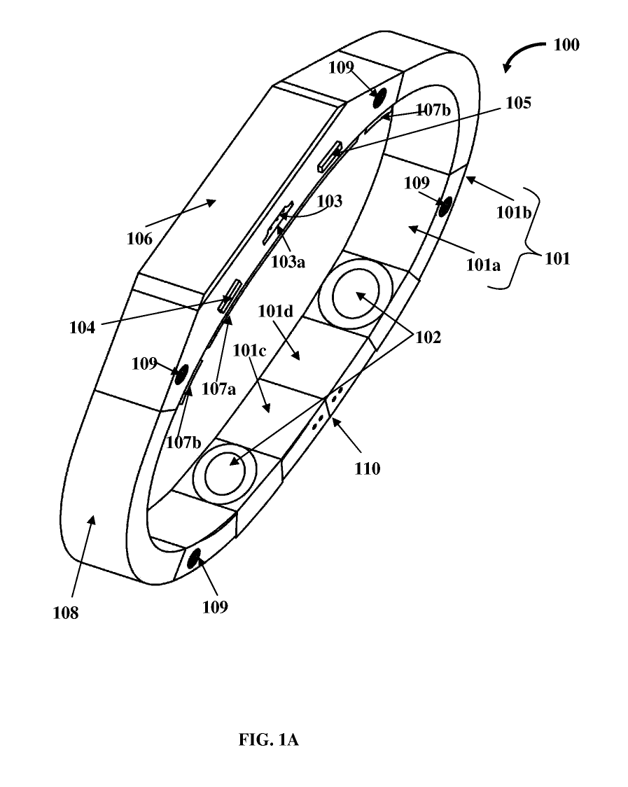

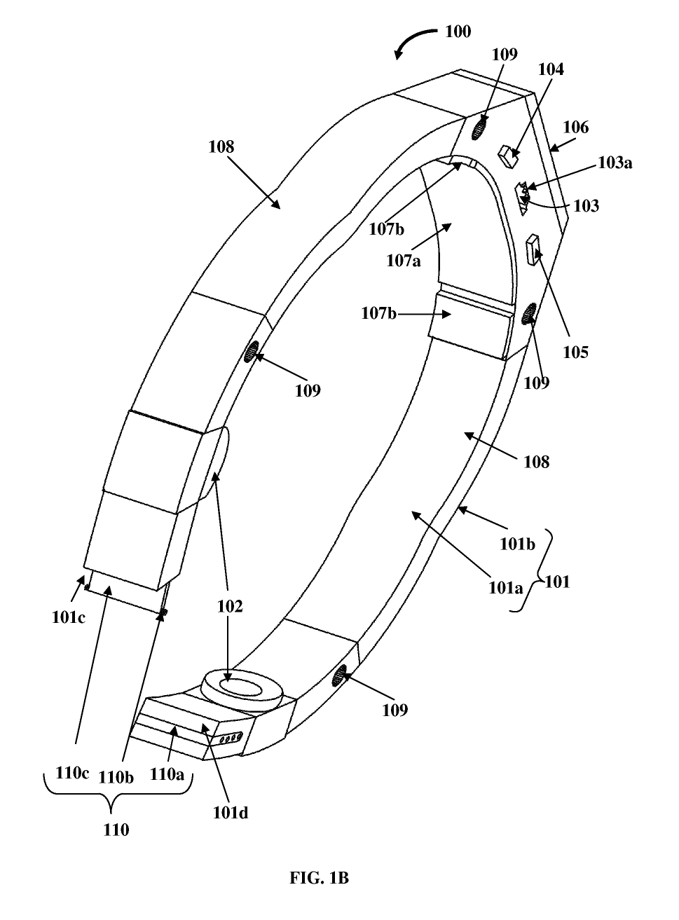

FIGS. 1A-1B exemplarily illustrate perspective views of a portable monitoring device 100. The portable monitoring device 100 is used for monitoring, detecting, and notifying physiological data associated with physiological parameters of a patient for indicating, for example, specific medical conditions and/or general health of the patient. As used herein, the term “patient” refers to a person or any user, for example, an adult, a child, an infant belong to one of various age groups, who require monitoring and alerting of their physiological parameters for specific medical care and/or general health care. The physiological parameters of the patient comprise, for example, heartbeat of the patient, electrocardiograph (ECG) of the patient, temperature of the patient, perspiration of the patient, etc. The portable monitoring device 100 detects and monitors the medical condition and vitals of the patient at all times and alerts the medical condition of the patient during undesired variations in the monitored physiological data associated with the physiological parameters of the patient.

The portable monitoring device 100 disclosed herein comprises a housing 101 made from a variety of materials, for example, metals such as platinum, polymers, etc. The housing 101 of the portable monitoring device 100 is configured in different shapes and configurations to resemble, for example, a watch face, a fashion accessory, a garment wearable on a patient's body part such as a wrist, an arm, etc. In an embodiment, the housing 101 is accessorized by adding outer shells or face-plates that resemble, for example, a watch face or a fashion accessory, etc., to cover the housing 101 of the portable monitoring device 100.

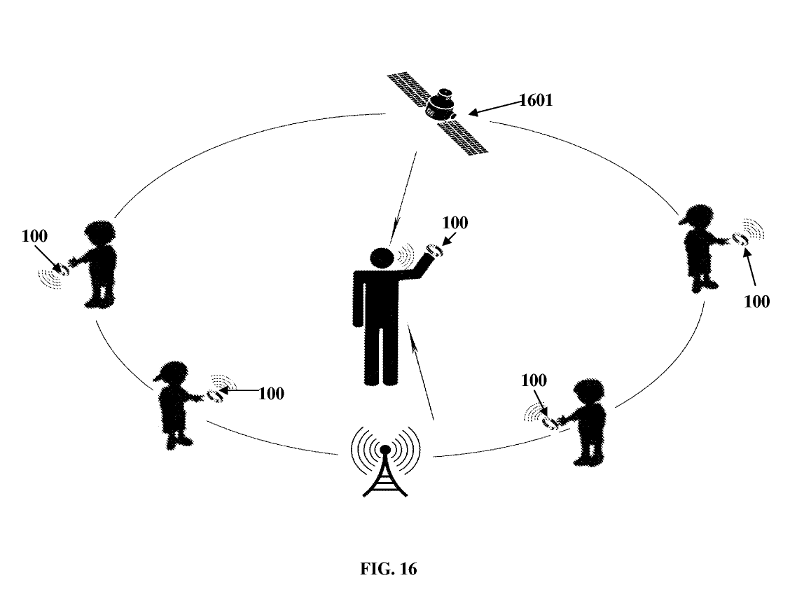

For purposes of illustration, the application of the portable monitoring device 100 refers to monitoring, detection and notification of physiological data associated with physiological parameters of a patient. However the scope of the portable monitoring device 100 is not limited to monitoring, detection, and notification of physiological data, but can be extended to include applications such as confining a patient within a boundary by applying a lock on the patient, reading blood glucose test strips, storing and playing back digital audio media using headphones, storing and playing back digital video media, wirelessly connecting to automotive services and transmitting automotive data to a mechanic, etc. The application of the portable monitoring device 100 may also be extended to functioning as a master or a host portable monitoring device 100 in conjunction with other portable monitoring devices 100 working in a master-slave relationship to transmit proximity data and for emergency alert functions as exemplarily illustrated in FIG. 16. The portable monitoring device 100 also monitors its battery life and internal components, contact points with the patient's body part, etc.

In an embodiment, the portable monitoring device 100 ensures correct administration of medical therapies, for example, medication, planned diagnostic studies or interventions such as computed tomography (CT) scan, magnetic resonance imaging (MRI), etc., resulting in a decrease of medical error and patient morbidity and mortality, thereby making the portable monitoring device 100 cost effective.

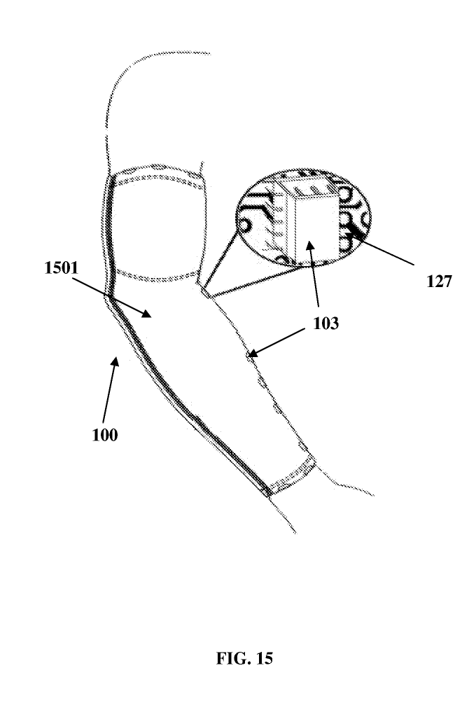

The housing 101 of the portable monitoring device 100 is configured to conform to a patient's body part. The patient's body part is, for example, a wrist or an arm of the patient. For purposes of illustration, the detailed description refers to the housing 101 being configured as a wrist band as exemplarily illustrated in FIGS. 1A-14C and a sleeve garment 1501 as exemplarily illustrated in FIG. 15, however the scope of the portable monitoring device 100 is not limited to a wrist band and a sleeve garment 1501, but may be extended to be configured into any wearable item that may be worn on and encases the hands, fingers, thumbs, toes, feet, head, elbows, or any other suitable body part of the patient's body for purposes of convenience and functionality.

The housing 101 of the portable monitoring device 100 defines an inner surface 101a for establishing physical contact with the patient's body part, and an outer surface 101b opposing the inner surface 101a. The housing 101 encloses the components of the portable monitoring device 100, for example, one or more parameter sensing devices 102, 107a, 107b, etc., data communication interfaces 103a, a processing unit 127, etc. The housing 101 of the portable monitoring device 100 is configured, for example, as a metallic bracelet as exemplarily illustrated in FIGS. 1A-1B. The housing 101 of the portable monitoring device 100 is made, for example, from materials such as platinum metal resistant to heat, water, shock, physical damage, etc. A patterned engraving is provided on the outer surface 101b of the housing 101 of the portable monitoring device 100 for enabling a user or a responder, for example, from an emergency medical service facility to identify the portable monitoring device 100 in case of an emergency medical condition of the patient. The housing 101 of the portable monitoring device 100 is strategically engineered to accommodate all the physical and technical aspects required for the overall functionality of the portable monitoring device 100.

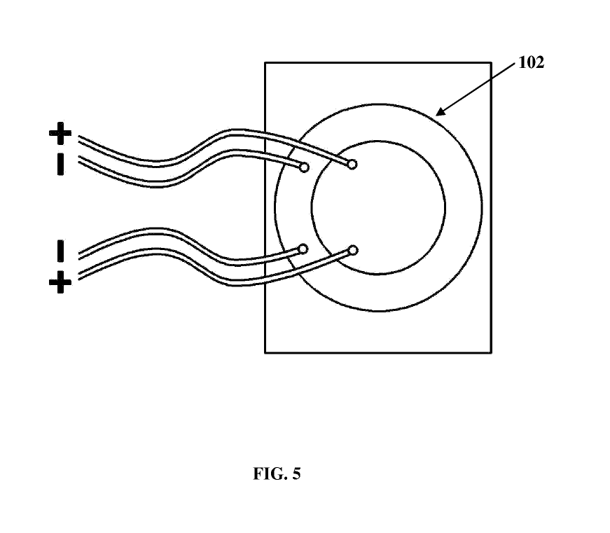

The portable monitoring device 100 disclosed herein further comprises one or more parameter sensing devices 102, 107a, 107b, etc., located at predetermined positions on the inner surface 101a of the housing 101 for acquiring the physiological data associated with the physiological parameters of the patient as disclosed in the detailed description of FIG. 5.

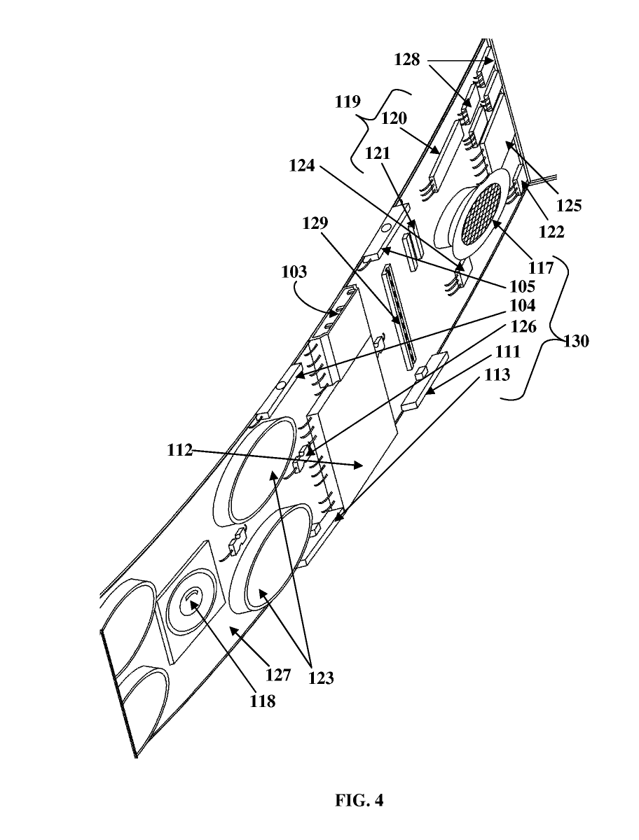

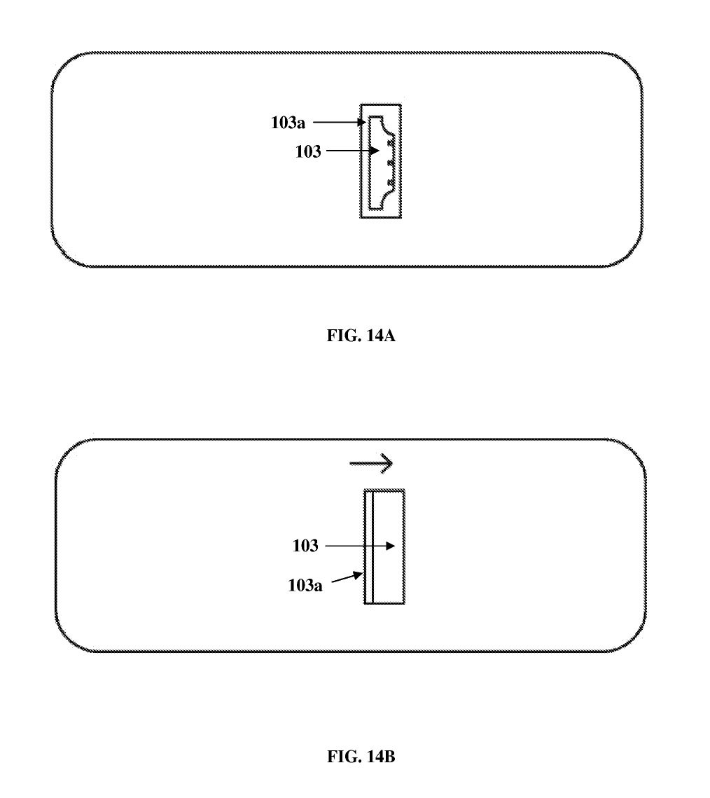

The portable monitoring device 100 disclosed herein further comprises a processing unit 127 disposed within the housing 101 and in communication with the parameter sensing devices 102, 107a, 107b, etc., for processing the acquired physiological data and patient information from the parameter sensing devices 102, 107a, 107b, etc., as disclosed in the detailed description of FIG. 4. The portable monitoring device 100 further comprises one or more data communication units 103 disposed within the housing 101 and in communication with the processing unit 127 for transmitting the processed physiological data and the patient information to one or more of a base monitoring unit 1700, a remote monitoring station 1803, and external responder devices 1801 as disclosed in the detailed description of FIGS. 14A-14B, FIGS. 17A-17D, and FIG. 18.

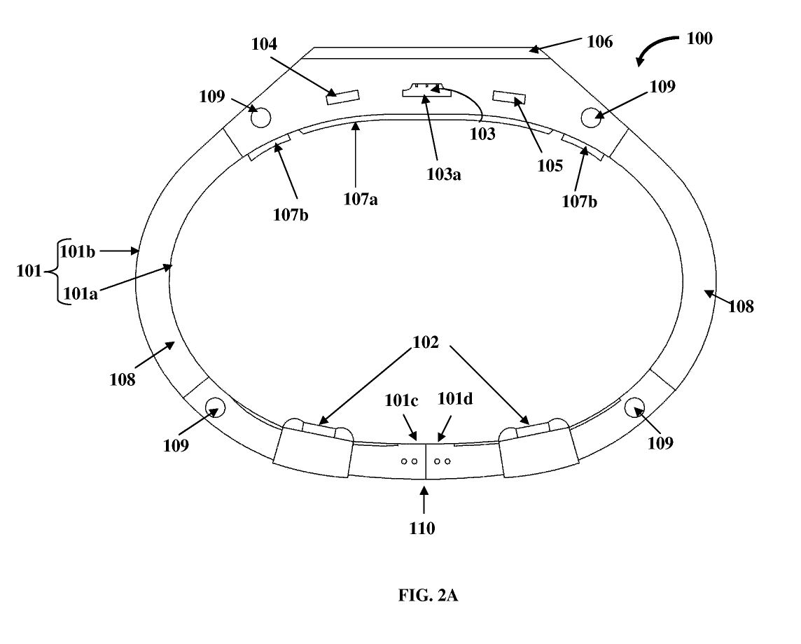

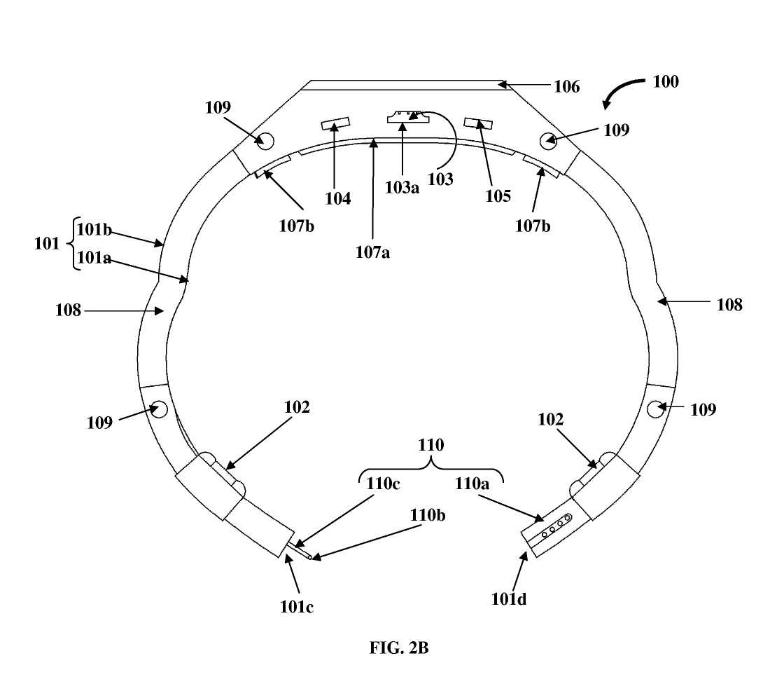

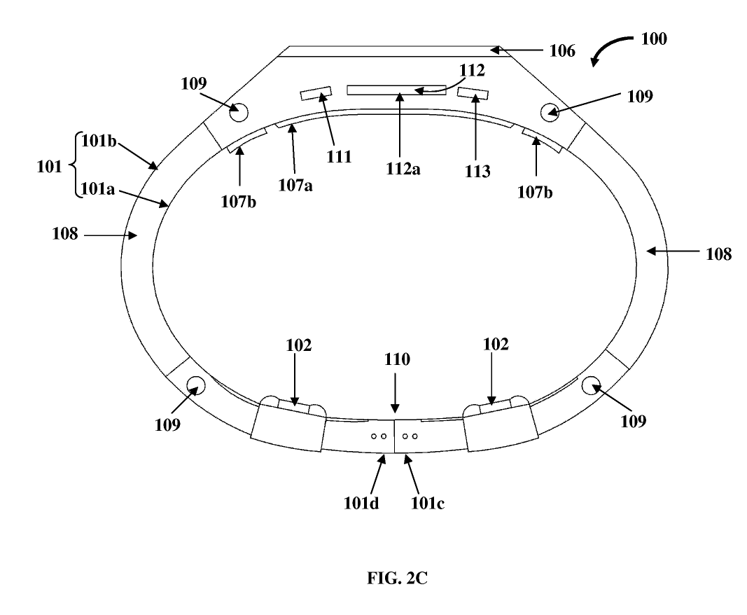

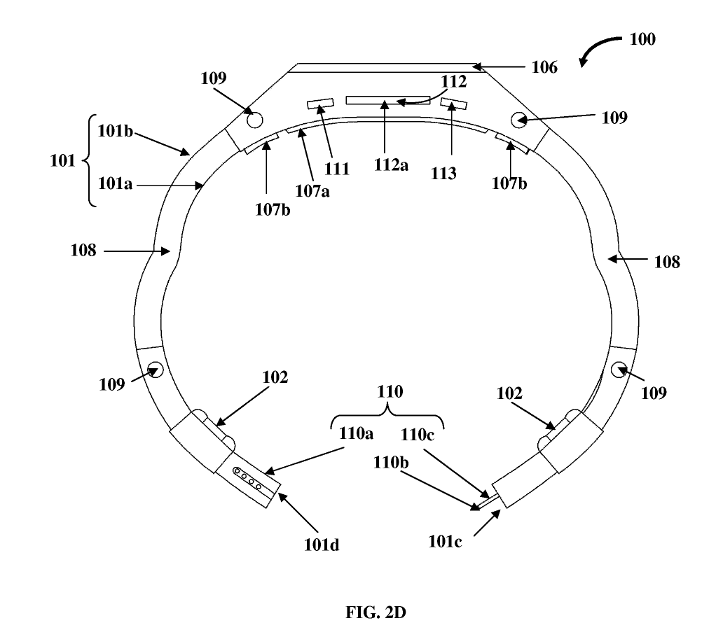

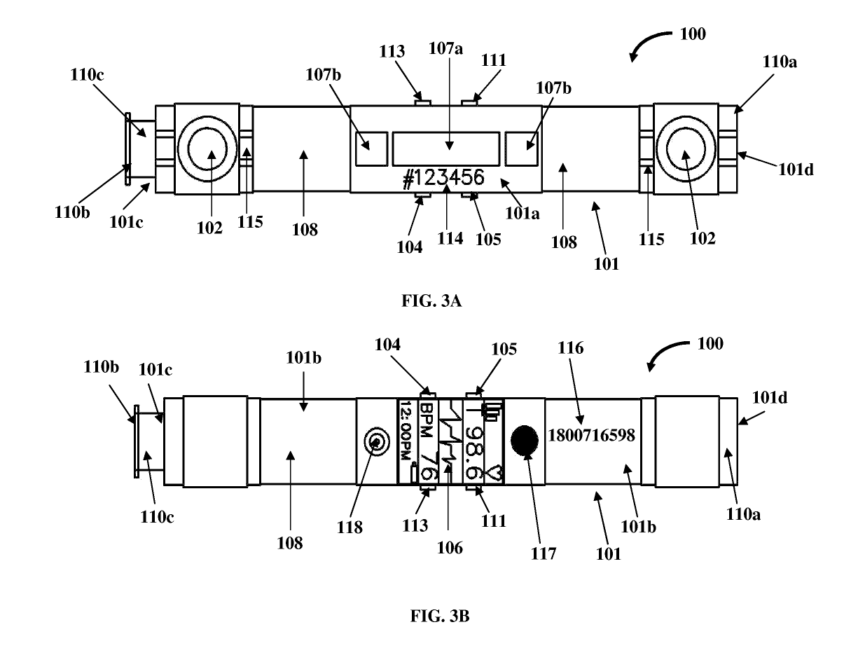

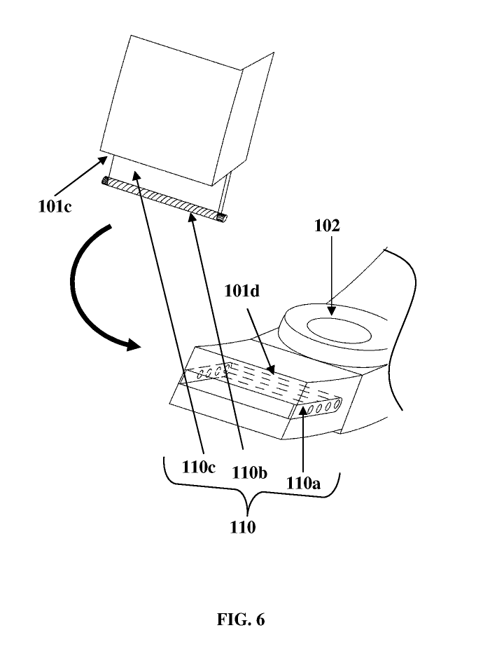

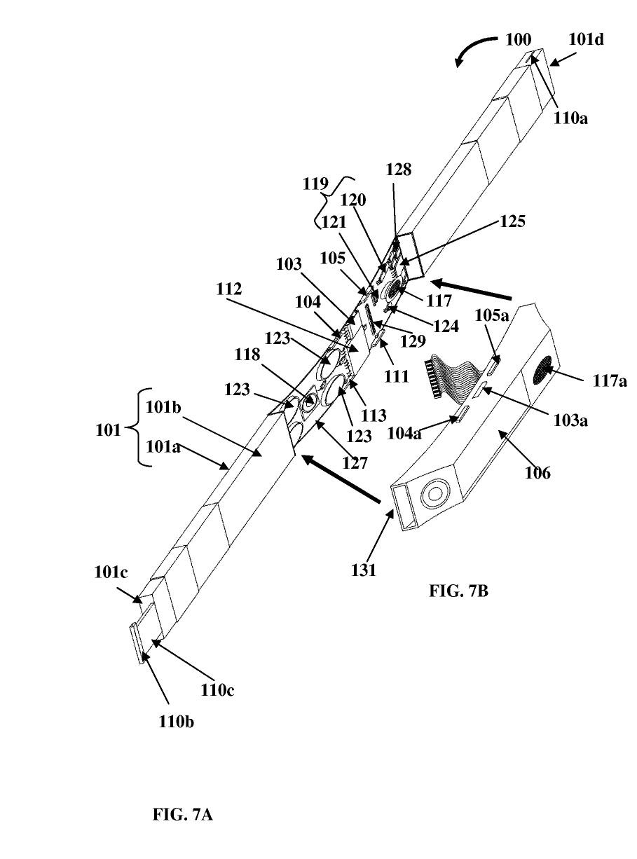

The portable monitoring device 100 disclosed herein further comprises user interface elements 104 and 105, for example, a marker button 104 and a function button 105 located at predetermined positions on the housing 101 as disclosed in the detailed description of FIGS. 2A-2B. The portable monitoring device 100 further comprises a liquid crystal display 106 as disclosed in the detailed description of FIG. 7B, and temperature sensing plates 107a and perspiration reader plates 107b as disclosed in the detailed description of FIG. 3A and FIG. 5. The housing 101 of the portable monitoring device 100 further comprises a rubber strapping 108 for allowing greater stretch ability to enable the portable monitoring device 100 to obtain an optimal grip on the patient's body part. The rubber strapping 108 is connected to each other by a faceplate connection point 109. The faceplate connection point 109 also enables attachment of the portable monitoring device 100 to one or more outer shells or accessories. The portable monitoring device 100 further comprises a fastening and resizing assembly 110 as disclosed in the detailed description of FIG. 6.

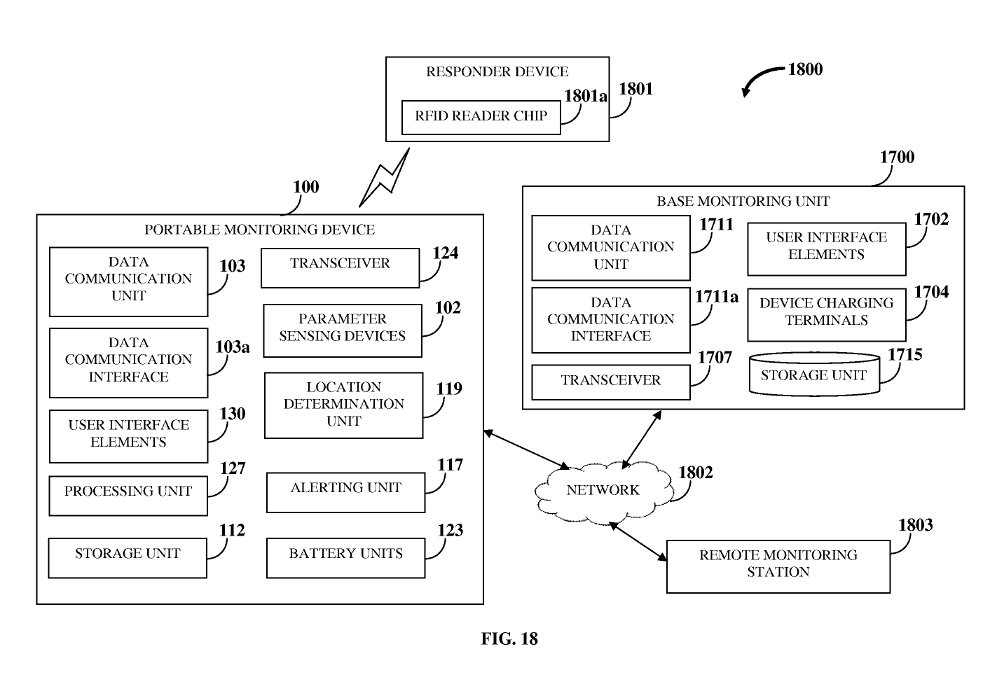

In an embodiment, the portable monitoring device 100 enables a remote monitoring station 1803, as exemplarily illustrated in FIG. 18, to determine a critical condition and dispatch assistance or authorities, for example, a fire department, a police department, etc., to the patient at a moment's notice. The portable monitoring device 100 can also alert multiple non-medical and emergency personnel such as family, friends, etc.

In another embodiment, the portable monitoring device 100 provides the ability to monitor the location and condition of aged and infirm adults at assisted living facilities where it is difficult for the staff to monitor every patient at all times. The portable monitoring device 100 allows internal monitoring of vitals, managing medication distribution, dosages, interactions with other medication, etc.

In an embodiment, the portable monitoring device 100 enables monitoring the condition of patients and storing physiological data associated with the physiological parameters of patients involved in drug trials and clinical research at all times. Physicians and researchers are notified instantly of any changes in the health of the patient associated with the portable monitoring device 100. In another embodiment, physicians may provide high risk patients with the portable monitoring device 100 to monitor the patients' health at all times and be alerted instantly of health issues. The portable monitoring device 100 is programmed to maintain high priority data for health care providers. In another embodiment, hospitals and physicians may monitor the recovery and health of the patient for a period of time following a surgical procedure to detect and prevent post-surgical complications. In another example, the portable monitoring device 100 enables monitoring of metabolic rate of a person to assist in physical training and weight loss.

FIGS. 2A-2B exemplarily illustrate right side orthogonal views of the portable monitoring device 100. The portable monitoring device 100 disclosed herein comprises the housing 101 defining an inner surface 101a and an outer surface 101b as disclosed in the detailed description of FIGS. 1A-1B, one or more parameter sensing devices 102, 107a, 107b, etc., as disclosed in the detailed description of FIG. 5, and one or more data communication interfaces 103a as disclosed in the detailed description of FIGS. 14A-14B The portable monitoring device 100 further comprises user interface elements 130 such as a marker button 104 and a function button 105. The marker button 104 is used to mark a time stamp associated with a health event. For example, the patient may depress the marker button 104 on the onset of a chest pain to identify the time at which the pain was first felt. The marker button 104 is also used to mark the time stamp when medication is provided to the patient. The function button 105 is used to turn on and turn off the functionality of the portable monitoring device 100. The function button 105 is used to change a mode of operation of the portable monitoring device 100. For example, the portable monitoring device 100 can be changed to an exercise mode when a patient is exercising to monitor and compare the physiological parameters of the patient with baseline parameters that are different from the baseline parameters maintained during monitoring and alerting physiological parameters of the patient.

FIGS. 2C-2D exemplarily illustrate left side orthogonal views of the portable monitoring device 100. The portable monitoring device 100 disclosed herein further comprises one or more temperature sensing plates 107a and perspiration reader plates 107b as disclosed in the detailed description of FIG. 3A and FIG. 5. The portable monitoring device 100 further comprises a liquid crystal display 106 as disclosed in the detailed description of FIGS. 7A-7B. The housing 101 of the portable monitoring device 100 comprises other user interface elements 130, for example, a panic button 111 and a reset button 113. The panic button 111 is used to send a panic alert to the remote monitoring station 1803. For example, the patient depresses the panic button 111 for sending out an alert to the remote monitoring station 1803 in addition to an audible shriek to anyone within a hearing distance of the patient. In an emergency, the patient or any other person assisting the patient can use the panic button 111 to alert the remote monitoring station 1803 of the emergency condition of the patient. The reset button 113 resets the function of the portable monitoring device 100. In an embodiment, the buttons 111, etc., on the portable monitoring device 100 are used for generating both silent and audible alarms. For example, when the patient presses the panic button 111, the portable monitoring device 100 sends a silent panic alarm to the remote monitoring station 1803 and an audible alarm to anyone in the hearing distance. The portable monitoring device 100 further comprises a storage unit 112 accessible via a storage unit slot 112a as disclosed in the detailed description of FIG. 4.

FIG. 3A exemplarily illustrates a bottom orthogonal view of the portable monitoring device 100, showing an inner surface 101a defined by the housing 101 of the portable monitoring device 100. The portable monitoring device 100 comprises temperature sensing plates 107a disposed on the inner surface 101a of the housing 101 for monitoring the temperature of the patient at all times. The temperature sensing plates 107a are in contact with the patient's body part to monitor the temperature of the patient. The portable monitoring device 100 further comprises perspiration reader plates 107b disposed on the inner surface 101a of the housing 101. The perspiration reader plates 107b are in contact with the patient's body part to monitor the perspiration of the patient. The portable monitoring device 100 further comprises a rubber strapping 108 as disclosed in the detailed description of FIGS. 1A-1B.

The portable monitoring device 100 further comprises a fastening and resizing assembly 110 comprising a multi-position clasp 110a, an insert 110b, and an extensible sliding member 110c as disclosed in the detailed description of FIG. 6. The portable monitoring device 100 further comprises a unique identifier 114 provided on the inner surface 101a of the housing 101 of the portable monitoring device 100 for identifying the patient associated with the portable monitoring device 100 and/or for logging the acquired physiological data and the patient information into a medical account of the patient for retrieval of the acquired physiological data and the patient information. In an embodiment, the unique identifier 114 is provided on the outer surface 101b of the housing 101 of the portable monitoring device 100. The portable monitoring device 100 further comprises multi-position tracks 115. The multi-position tracks 115 position the parameter sensing devices 102, 107a, and 107b on the housing 101. The parameter sensing devices 102, 107a, and 107b pick up optimum readings associated with the physiological parameters of the patient as disclosed in the detailed description of FIG. 5.

FIG. 3B exemplarily illustrates a top orthogonal view of the portable monitoring device 100, showing an outer surface 101b defined by the housing 101 of the portable monitoring device 100. The portable monitoring device 100 further comprises emergency contact information 116 of, for example, one or more emergency contacts provided on the outer surface 101b of the housing 101 of the portable monitoring device 100 for enabling provision of relief measures to the patient by reaching emergency responders. In an embodiment, the emergency contact information 116 is provided on the inner surface 101a of the housing 101 of the portable monitoring device 100. The emergency contact information 116 comprises, for example, a hotline number of an emergency department of a nearby hospital, the number of the police department, the fire department, etc. The emergency contact information 116 enables the first responders or any other person assisting the patient requiring emergency medical attention to dial the hotline number of the emergency department of the hospital. The emergency contact information 116 also comprises a list of contacts, numbers to alert other individuals wearing the portable monitoring device 100, etc.

The portable monitoring device 100 further comprises an alerting unit 117 and an image capture device 118 as disclosed in the detailed description of FIG. 4. The portable monitoring device 100 comprises an extensible sliding member 110c, an insert 110b, and a multi-position clasp 110a for fastening and resizing the portable monitoring device 100 on the patient's body part. The portable monitoring device 100 further comprises the function button 105, the marker button 104, the panic button 111, the reset button 113, and the alerting unit 117 for providing the user with audio visual input/output.

FIG. 4 exemplarily illustrates an internal perspective view of the portable monitoring device 100, showing internal components of the portable monitoring device 100. The portable monitoring device 100 comprises one or more data communication units 103 accessible via one or more data communication interfaces 103a, the marker button 104, and the function button 105 as disclosed in the detailed description of FIGS. 2A-2B, and the panic button 111 and the reset button 113 as disclosed in the detailed description of FIGS. 2C-2D. The portable monitoring device 100 further comprises a storage unit 112 in communication with the processing unit 127 and disposed within the housing 101 for storing the acquired physiological data and the patient information and for providing backup of the acquired physiological data and the patient information. The patient information comprises, for example, health information of the patient, billing information of the patient, insurance information of the patient, historical patient information, and any combination thereof. The storage unit 112 is external and/or internal to the portable monitoring device 100. The storage unit 112 is, for example, a secure digital memory card or any other memory storage device. The storage unit 112 is, for example, a quarter the size of a standard sized secure digital memory card.

The storage unit 112 is inserted into a storage unit slot 112a, for example, a secure digital card slot for storing the physiological data acquired by the parameter sensing devices 102, 107a, 107b, etc. The portable monitoring device 100 stores the physiological data associated with the physiological parameters, for example, heart rate, pulse rate, temperature, etc., of the patient in the storage unit 112 and transmits the physiological data of the patient to the base monitoring unit 1700 and the remote monitoring station 1803 periodically. For example, physicians can instantly review the status of the patient from a past time period by accessing the physiological data stored in the storage unit 112. The patient may anytime access the physiological data and the patient information stored on the storage unit 112. Furthermore, the periodic backup of the physiological data in the storage unit 112 and the remote monitoring station 1803 enables the patient to review and understand the key functions of the body based on the stored physiological data. The remote monitoring station 1803 also periodically updates a patient history log stored in the remote monitoring station 1803 based on the acquired physiological data of the patient.

The portable monitoring device 100 disclosed herein further comprises an alerting unit 117 located at predetermined positions on either the outer surface 101b of the housing 101 or within the housing 101 for remotely alerting the remote monitoring station 1803 or locally alerting persons in close proximity to the patient, of a medical condition of the patient, and for providing an audible alert to persons in the vicinity of the patient, for example, via an audio mode, a visual mode, and any combination thereof. The alerting unit 117 is in communication with the processing unit 127 of the portable monitoring device 100. In an embodiment, the alerting unit 117 alerts about the medical condition of the patient through the user interface elements 130 of the portable monitoring device 100, for example, a speaker that is incorporated in the housing 101 of the portable monitoring device 100 to transmit an audible alert. In an emergency medical situation of the patient, the alerting unit 117 generates an audible shriek to alert people around the patient to assist the patient. Furthermore, the alerting unit 117 sends, for example, an emergency alert to the remote monitoring station 1803 for the remote monitoring station 1803 to dispatch emergency responders for initiating relief measures to the patient. The alerting unit 117 provides remote alerts and/or local alerts when the acquired physiological data associated with the physiological parameters of the patient exceeds baseline parameter values.

The user interface elements 130 comprise, for example, the marker button 104, the function button 105, the panic button 111, the reset button 113, the alerting unit 117, light emitting diodes 126, etc. The user interface elements 130 further comprise, for example, a one way speaker, a two way speaker, a microphone, etc., and a combination thereof. One or more of the user interface elements 130 provide an audiovisual indication of the functioning of the portable monitoring device 100, power levels of the portable monitoring device 100, disconnection of the parameter sensing devices 102, 107a, 107b, etc., operating status of the portable monitoring device 100, etc. In an embodiment, the user interface elements 130 allow personnel at the remote monitoring station 1803 to speak to the patient and allow the patient to respond back. In another embodiment, the alerting unit 117 generates and transmits a visual and/or audio indicator in the event of catastrophic occurrences such as natural disasters, terrorist attacks, etc. In another embodiment, the alerting unit 117 generates an alert for organ transplant readiness. In the event that the patient is awaiting an organ transplant, the alerting unit 117 alerts a patient when an organ is available. Conversely, the portable monitoring device 100 worn by an organ donor can automatically update and alert a hospital of their available organs upon the donor's demise.

In an embodiment, the portable monitoring device 100 provides an image capture device 118, for example, a camera, for capturing images of the patient. For example, the portable monitoring device 100 captures audio and video of the patient at all times or at periodic intervals and stores the audio and video in the storage unit 112. The captured audio and video may also be transmitted to the remote monitoring station 1803 via a network 1802. The image capture device 118 may also be configured as a quick response (QR) code or matrix barcode reader to identify, for example, medications, etc.

The portable monitoring device 100 further comprises a location determination unit 119 disposed within the housing 101 and in communication with the processing unit 127 for determining geographic location of the patient. The location determination unit 119 comprises a location determination device 120, for example, a global positioning system, and a location determination antenna 121, for example, a global positioning system antenna. The location determination unit 119 locates the patient for dispatching the emergency responders to the patient when a relief measure is required.

The location determination unit 119 enables the portable monitoring device 100 to pinpoint the exact location of the patient to provide immediate medical attention whenever a medical emergency situation arises with the patient. The location determination unit 119 calculates the position of the portable monitoring device 100 by timing multiple signals sent by a constellation of location determination satellites above the earth, as is known in the art. The location determination antenna 121 receives location determination signals from location determination satellites and carries the location determination signals to the location determination device 120 of the location determination unit 119 for determining the location information. The location determination unit 119 transmits the location information to the remote monitoring station 1803. The remote monitoring station 1803 immediately passes the location information to one or more external responder devices 1801 associated with the responders. The location determination unit 119 can be turned off and on as and when required. The location determination unit 119 is turned on automatically in case of an emergency alert. The location determination unit 119 can also work in a master slave relationship.

The portable monitoring device 100 comprises a radio frequency identification (RFID) chip 122 that allows identification and tracking of the portable monitoring device 100 using radio frequency. The RFID chip 122 comprises, for example, an RFID tag which may be scanned from several meters away and beyond the line of sight of an RFID reader, thereby allowing an emergency responder to connect and synchronize a hand held responder device 1801 to the portable monitoring device 100 in order to receive the acquired physiological data, the patient information, and all the medical information at the site of the patient as well as to triangulate the patient's location. The RFID communication enables the first responder to sync up with the portable monitoring device 100 in case of an emergency and retrieve the required data prior to treatment of the patient. Using the RF transceiver 124 or a satellite antenna herein also represented by the reference numeral 124, the portable monitoring device 100 can also link up with a designated cellular provider to relay information through their network.

The portable monitoring device 100 further comprises one or more battery units 123 located inside the housing 101 of the portable monitoring device 100 for powering the portable monitoring device 100. Each battery unit 123 is, for example, a silver oxide battery. The battery units 123 also transmit power to the light emitting diodes 126 of the portable monitoring device 100. The battery units 123 have a higher circuit potential than a mercury battery and a flatter discharge curve than a standard alkaline battery.

The satellite antenna 124 provides the portable monitoring device 100 with better connectivity and coverage during communication by allowing stronger signal strength. The satellite antenna 124 uses radio frequency modules for high speed data transmission. For example, micro-electronic circuits in a digital radio frequency architecture operate at speeds of up to 100 gigahertz (GHz). The satellite antenna 124 comprises software programmable digital processors that permit conversion between digital baseband signals and analog radio frequency.

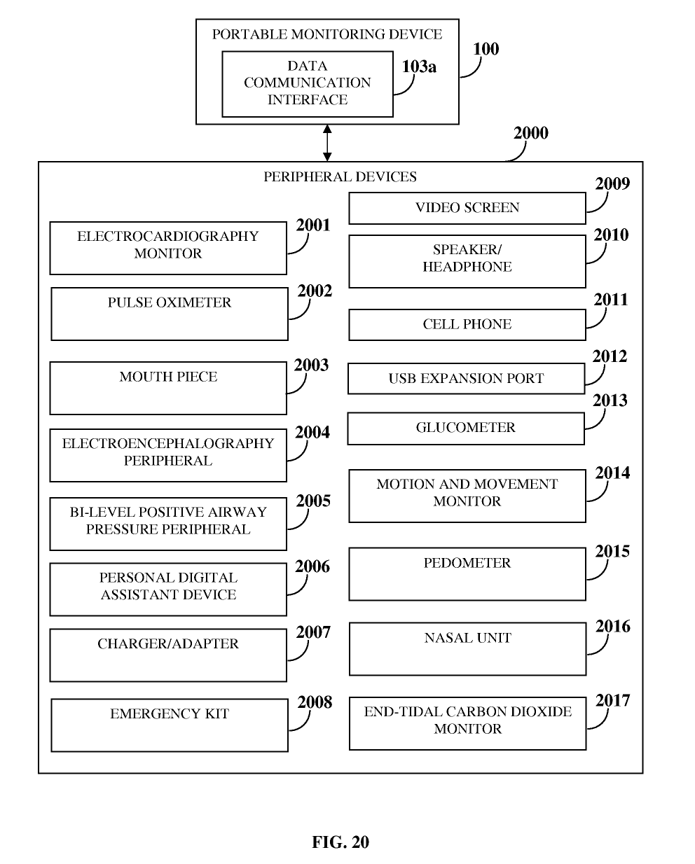

The portable monitoring device 100 provides for Bluetooth® communications using the data communication units 103. The data communication unit 103 of the portable monitoring device 100 comprises, for example, a Bluetooth chip for wireless communication with the base monitoring unit 1700, one or more peripheral devices 2000 as exemplarily illustrated in FIG. 20, and the responder devices 1801. In an embodiment, the portable monitoring device 100, communicates with the base monitoring unit 1700 via a Bluetooth chip 125 provided in the processing unit 127. The base monitoring unit 1700 communicates with the remote monitoring station 1803, for example, a call center at a remote location, using the RF transceiver 124 or the satellite antenna 124.

In an embodiment, the portable monitoring device 100 may monitor data collected from a pacemaker via the Bluetooth chip 125 using a Bluetooth communication protocol. In an embodiment, the portable monitoring device 100 may monitor data collected from a defibrillator implanted near the heart for detecting critical situations, via the Bluetooth chip 125. In another embodiment, the portable monitoring device 100 may be linked to an artificial heart technology via the Bluetooth chip 125. The portable monitoring device 100 comprises one or more microchips 128. The microchips 128 process the basic functions of the portable monitoring device 100. The microchips 128 process the external signals received from the satellite antenna 124, the location determination unit 119, the RFID chipset 122, etc. The microchips 128 process communication signals from the base monitoring unit 1700. The microchips 128 also comprise an extra backup microchip in case of a failure in any of the microchips 128.

The portable monitoring device 100 comprises one or more light emitting diodes 126 disposed at predetermined positions on the outer surface 101b of the housing 101 for providing a visual indication of the functioning of the portable monitoring device 100, power levels of the portable monitoring device 100, disconnection of the parameter sensing devices 102, 107a, 107b, etc., operating status of the portable monitoring device 100, status of the battery units 123 of the portable monitoring device 100, etc. The light emitting diodes 126 are, for example, semiconductor light emitting diodes (LEDs). The light emitting diodes 126 may indicate the transmission of the physiological data associated with the physiological parameters of the patient to the remote monitoring station 1803. The liquid crystal display 106 communicates with the processing unit 127 via the liquid crystal display interface 129 for providing a visual indication of the functioning of the portable monitoring device 100.

The portable monitoring device 100 comprises the processing unit 127 implemented, for example, on a flex motherboard. The processing unit 127 processes the acquired physiological data and the patient information. For example, the processing unit 127 processes heart rate output signals, ECG output signals, etc., acquired from the parameter sensing devices 102 and converts the acquired output signals, for example, from analog signals to digital signals to obtain the heart rate, ECG readings, etc. The processing unit 127 provides for back end software and programming for monitoring, tracking, dispatch and communications in response to processing the acquired physiological data associated with the physiological parameters of the patient. In an embodiment, the processing unit 127 is configured to flexibly adapt to the shape of the portable monitoring device 100 and is in communication with the components 102, 107a, 107b, etc., of the portable monitoring device 100. The processing unit 127 ties the communications and features of the portable monitoring device 100.

The motherboard or the circuit board of the processing unit 127 is either single-sided with one conductor layers or double sided with two conductor layers. The processing unit 127 is fabricated either with or without plated through holes. When the processing unit 127 is manufactured with plated through holes, terminations for the electronic components are provided on both sides of the circuit board, thus allowing the electronic components to be placed on either side of the circuit board. The double sided flex circuit boards are fabricated with protective cover layers on either one, or both or neither side of the fabricated circuit board.

The portable monitoring device 100 comprises an RF transceiver 124 that transmits the processed physiological data and the patient information to one or more of the base monitoring unit 1700, the remote monitoring station 1803, and the responder devices 1801, for example, in a wired mode of communication, a wireless mode of communication, or a combination thereof. For example, the RF transceiver 124 enables an uplink from the portable monitoring device 100 to a satellite. The portable monitoring device 100, the base monitoring unit 1700, the responder devices 1801, and the remote monitoring station 1803 communicate with each other wirelessly, for example, using the RF transceiver 124. The processed physiological data and the patient information transmitted to the remote monitoring station 1803 by the RF transceiver 124 are updated in a medical history log of the patient in the remote monitoring station 1803.

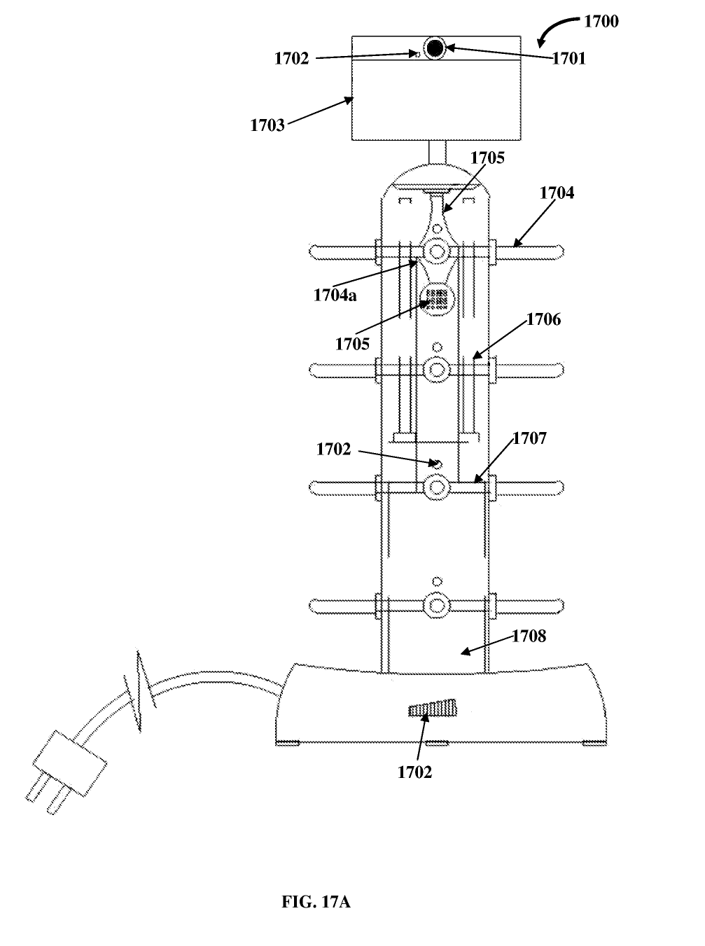

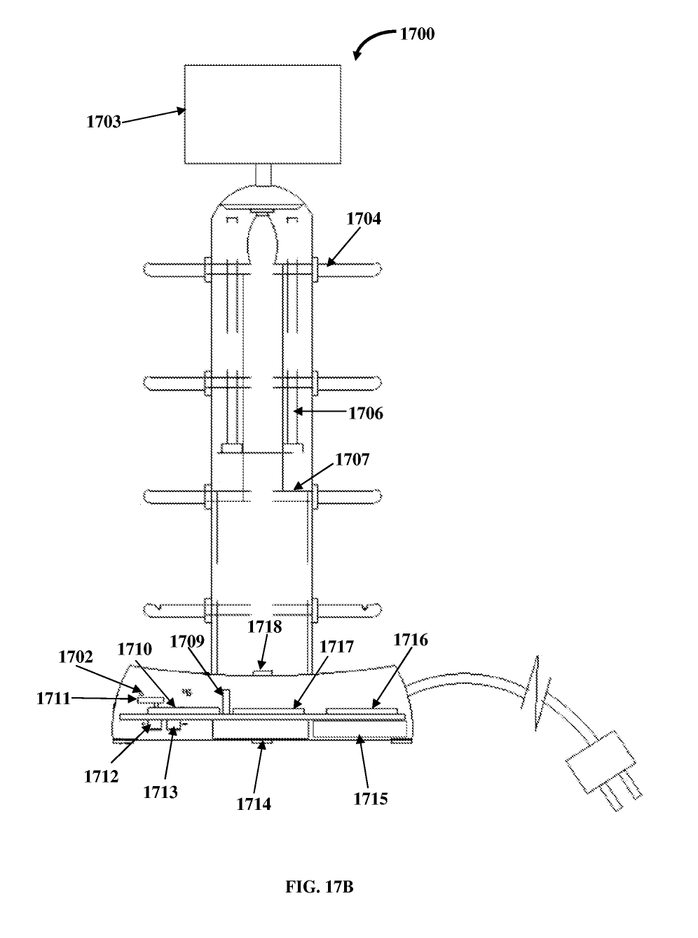

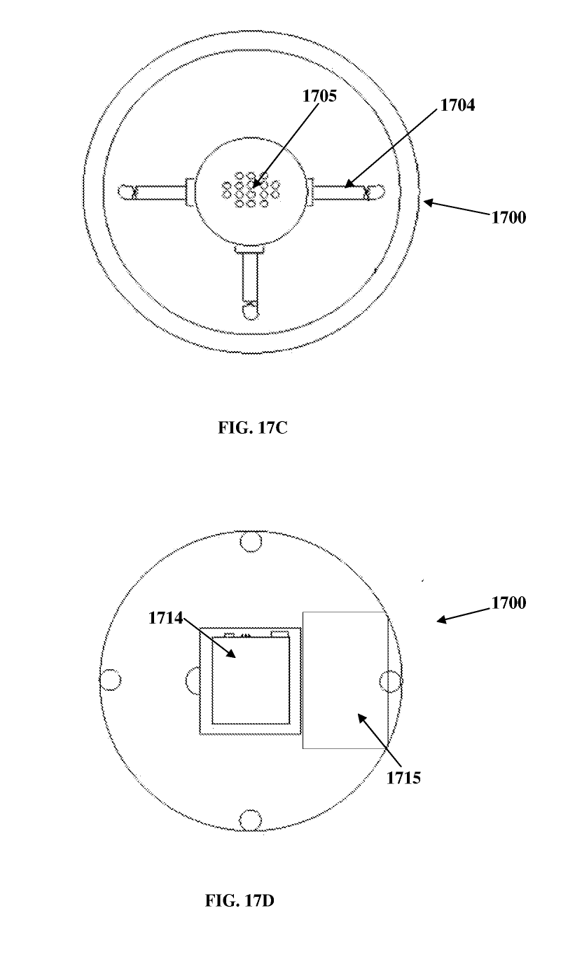

FIG. 5 exemplarily illustrates an orthogonal view of a parameter sensing device 102 of the portable monitoring device 100. One or more parameter sensing devices 102 are located at predetermined positions on the inner surface 101a of the housing 101 for acquiring the physiological data associated with physiological parameters of the patient by establishing physical contact with a patient's body part. As used herein, the term parameter sensing device 102, 107a, or 107b refers to any device that is used to collect physiological data associated with the physiological parameters of the patient. The parameter sensing devices 102, 107a, 107b, etc., are, for example, regulatory perspiration reader plates 107b, temperature sensing diodes or plates 107a, pulse beat sensing diodes 102, etc. The perspiration reader plates 107b collect and analyze the perspiration for multiple physiological conditions such as autoimmune diseases, diabetic autonomic neuropathy, reflex sympathetic dystrophy, drug intoxication, fertility, heart disease, genetic defects, etc. The pulse beat sensing diodes 102 monitor the heartbeat of the patient, the electrical signals from the heart, etc. The pulse beat sensing diodes 102 detect and amplify the tiny electrical changes on the skin that is caused due to the depolarization of the heart muscle during each heartbeat. The pulse beat sensing diodes 102 measure and diagnose abnormal rhythms of the heart, particularly abnormal rhythms caused by damage to the conductive tissue of the patient that carries electrical signals, or abnormal rhythms caused by electrolyte imbalances. The parameter sensing devices 102, 107a, 107b, etc., gather vitals of a patient on a continual basis. The gathered data is analyzed and stored in the storage unit 112 as disclosed in the detailed description of FIG. 4 and transmitted to the base monitoring unit 1700 as disclosed in the detailed description of FIGS. 17A-17B. In an embodiment, the parameter sensing devices 102 measure arterial flow via ultrasonic wave detection.

FIG. 6 exemplarily illustrates a fastening and resizing assembly 110 of the portable monitoring device 100 for fastening and resizing the portable monitoring device 100 on a patient's body part. The fastening and resizing assembly 110 of the portable monitoring device 100 comprises a multi-position clasp 110a, an insert 110b, and one or more extensible sliding members 110c. The extensible sliding member 110c is provided at one end 101c of the housing 101, while the multi-position clasp 110a is provided as the other end 101d of the housing 101. The insert 110b is a rod-shaped structure provided within the extensible sliding member 110c. The extensible sliding member 110c with the insert 110b is inserted into the multi-position clasp 110a to allow engagement of the insert 110b into the multi-position clasp 110a, thereby forming a loop around the wrist of the patient. The extensible sliding member 110c located at the end 101c of the housing 101 is configured for resizing the portable monitoring device 100 to conform to a size of the patient's body part and for positioning the parameter sensing devices 102, 107a, 107b, etc., on one or more touch points on the patient's body part. The extensible sliding member 110c allows repositioning of the parameter sensing devices 102, 107a, 107b, etc., on the housing 101 to pick up optimum readings associated with the physiological parameters of the patient.

The extensible sliding member 110c allows the portable monitoring device 100 to be resized by sliding the extensible sliding member 110c in or out. The multi-position clasp 110a, the insert 110b, and the extensible sliding member 110c interact with each other to secure the portable monitoring device 100 onto the patient's body part. The ability to resize the portable monitoring device 100 is centered around the need to precisely position and secure the parameter sensing devices 102, 107a, 107b, etc., on the touch points for monitoring the physiological data associated with the physiological parameters of the patient. The portable monitoring device 100 is available in multiple distinct sizes, for example, small, medium, large, and extra large. These distinct sizes of the portable monitoring device 100 are further adjustable to conform to the patient's body part.

FIGS. 7A-7B exemplarily illustrate a perspective view of the portable monitoring device 100, showing a detachable section 131 housing a liquid crystal display 106 that provides visual indications to the patient. The portable monitoring device 100 comprises the alerting unit 117, the image capture device 118, and the location determination unit 119 as disclosed in the detailed description of FIG. 4. The portable monitoring device 100 comprises the storage unit 112 and the liquid crystal display interface 129 as disclosed in the detailed description of FIG. 4.

The detachable section 131, as exemplarily illustrated in FIG. 7B, houses the liquid crystal display 106. The detachable section 131 can be removed from the housing 101 and reattached. The liquid crystal display 106 is a thin, flat electronic device that uses the light modulating properties of liquid crystals. The detachable section 131 comprises a marker button socket 104a for connecting the marker button 104 and a function button socket 105a for connecting the function button 105, and the data communication interface 103a for inserting the data communication unit 103. The detachable section 131 comprises an alerting unit housing 117a for enclosing the alerting unit 117. The liquid crystal display 106 screens enable parents to monitor an infant on the portable monitoring device 100 via a web camera or the image capture device 118 as disclosed in the detailed description of FIG. 4. The liquid crystal display 106 displays physiological data of a patient in real time, and on any compatible device via transmission of data from the portable monitoring device 100. The liquid crystal display 106 also displays information related to the portable monitoring device 100 and functions associated with the portable monitoring device 100.

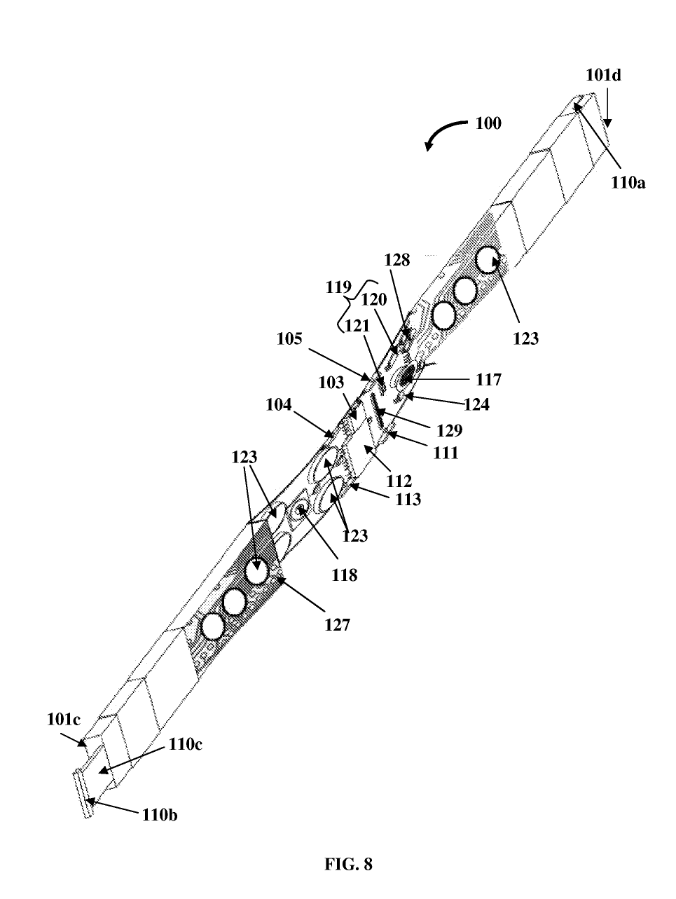

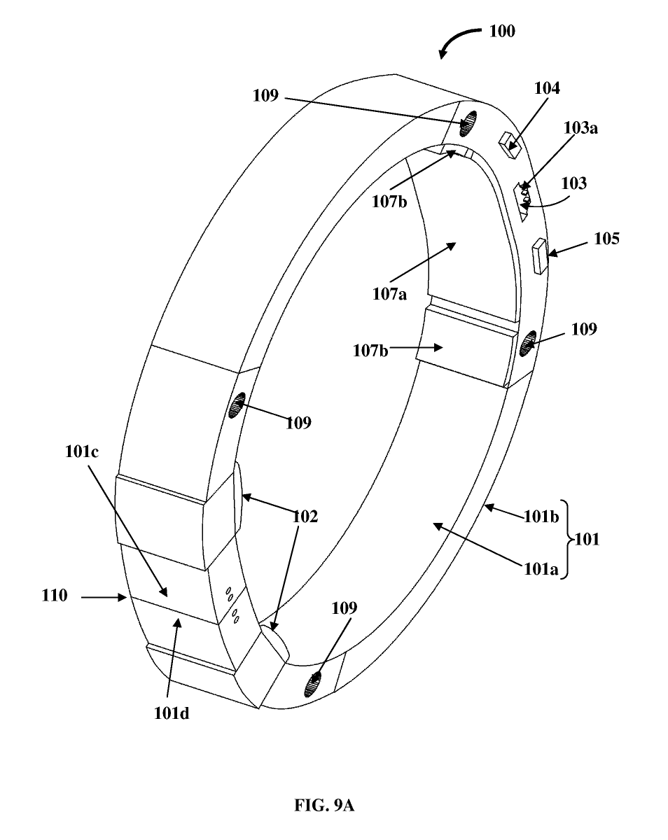

FIG. 8 exemplarily illustrates a perspective view of an embodiment of the portable monitoring device 100, showing multiple battery units 123 that provide additional power supply to the portable monitoring device 100. The portable monitoring device 100 comprises one or more battery units 123 disposed in the housing 101 of the portable monitoring device 100. The battery units 123 provide additional power supply to the portable monitoring device 100. The processing unit 127 of the portable monitoring device 100 runs across the rubber strapping 108 on the portable monitoring device 100. The battery units 123 power the liquid crystal display 106 as exemplarily illustrated in FIG. 7B, the peripheral devices 2000 as exemplarily illustrated FIG. 20, the image capture device 118, etc. The battery units 123 provide a backup power source for the portable monitoring device 100 for monitoring vitals of the patient and alerting the remote monitoring station 1803 of the physiological parameters of the patient.

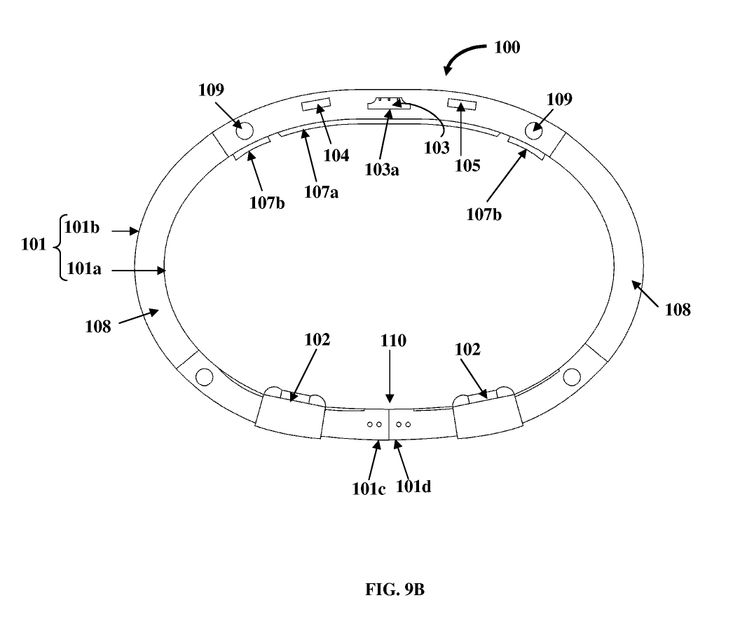

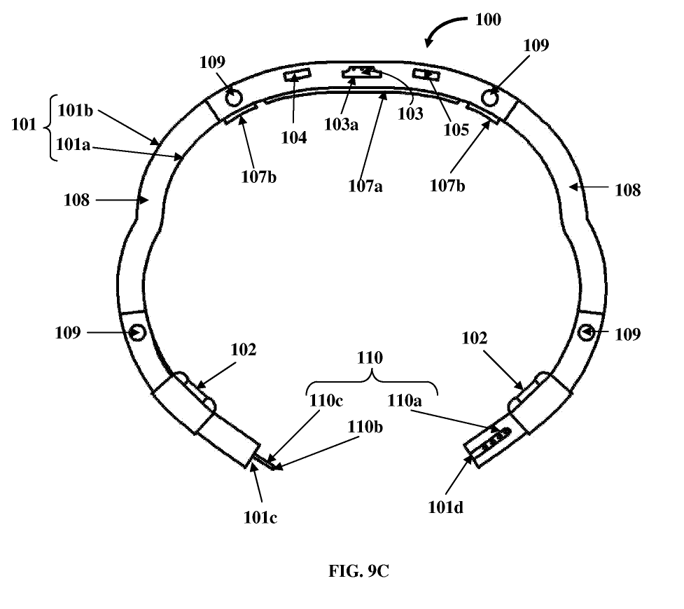

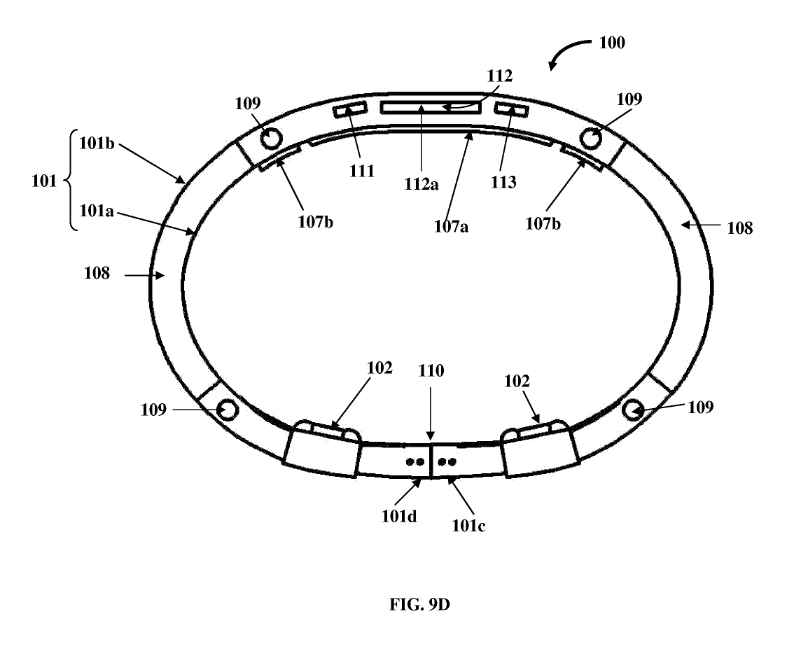

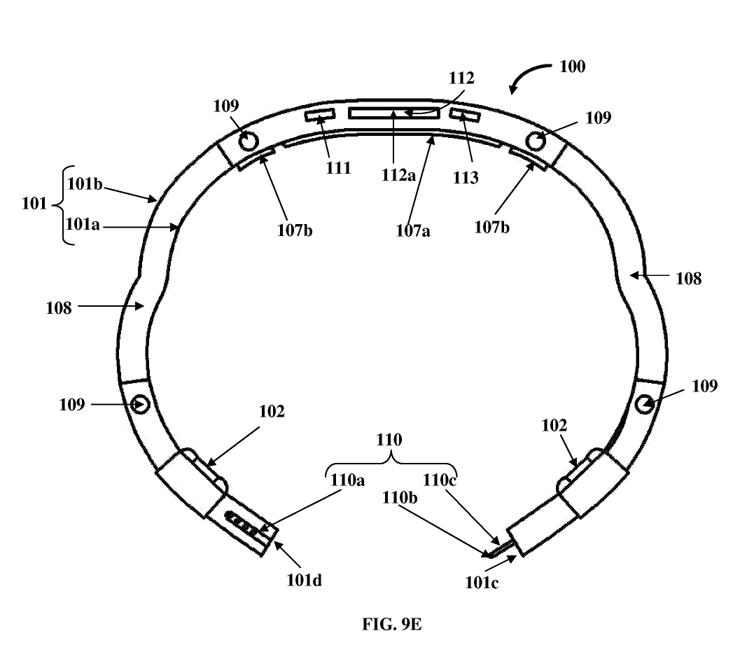

FIGS. 9A-9E exemplarily illustrate multiple views of an embodiment of the portable monitoring device 100. In this embodiment, the portable monitoring device 100 is free from a liquid crystal display 106 as compared to the embodiment of the portable monitoring device 100 illustrated in FIGS. 1A-1B. Right side orthogonal views of the portable monitoring device 100, showing the components 102, 103, 104, 105, 107a, 107b, 109, and 110 of the portable monitoring device 100 are exemplarily illustrated in FIGS. 9B-9C. Left side orthogonal views of the portable monitoring device 100, showing the components 102, 107a, 107b, 109, 110, 111, 112, and 113 are exemplarily illustrated in FIGS. 9D-9E.

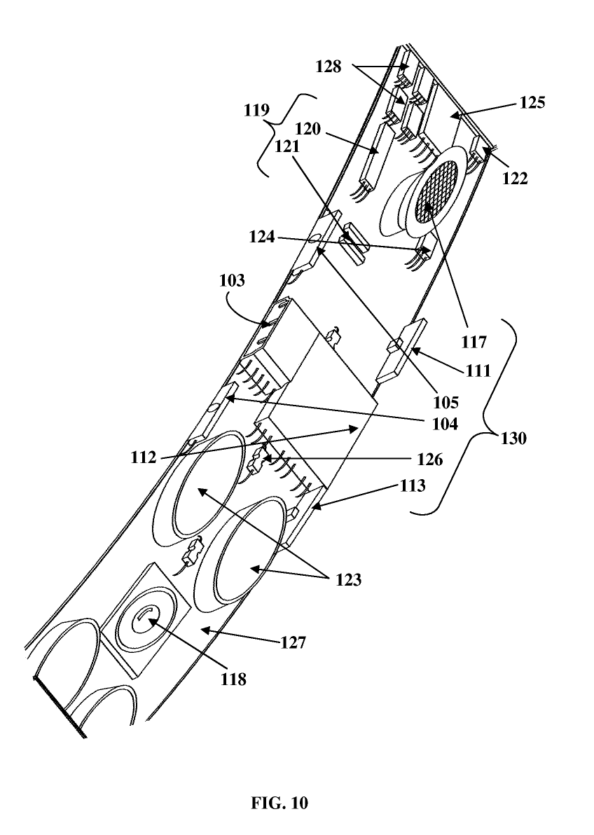

FIG. 10 exemplarily illustrates an internal perspective view of an embodiment of the portable monitoring device 100, showing the internal components of the portable monitoring device 100. The portable monitoring device 100 comprises a data communication unit 103 as disclosed in the detailed description of FIGS. 14A-14B, the marker button 104 and the function button 105 as disclosed in the detailed description of FIGS. 2A-2B, the panic button 111 and the reset button 113 as disclosed in the detailed description of FIGS. 2C-2D, the storage unit 112, the alerting unit 117, the image capture device 118, the location determination unit 119, the radio frequency identification chip 122, the battery units 123, the satellite antenna 124 or the RF transceiver 124, the Bluetooth chip 125, one or more light emitting diodes 126, and the processing unit 127 as disclosed in the detailed description of FIG. 4.

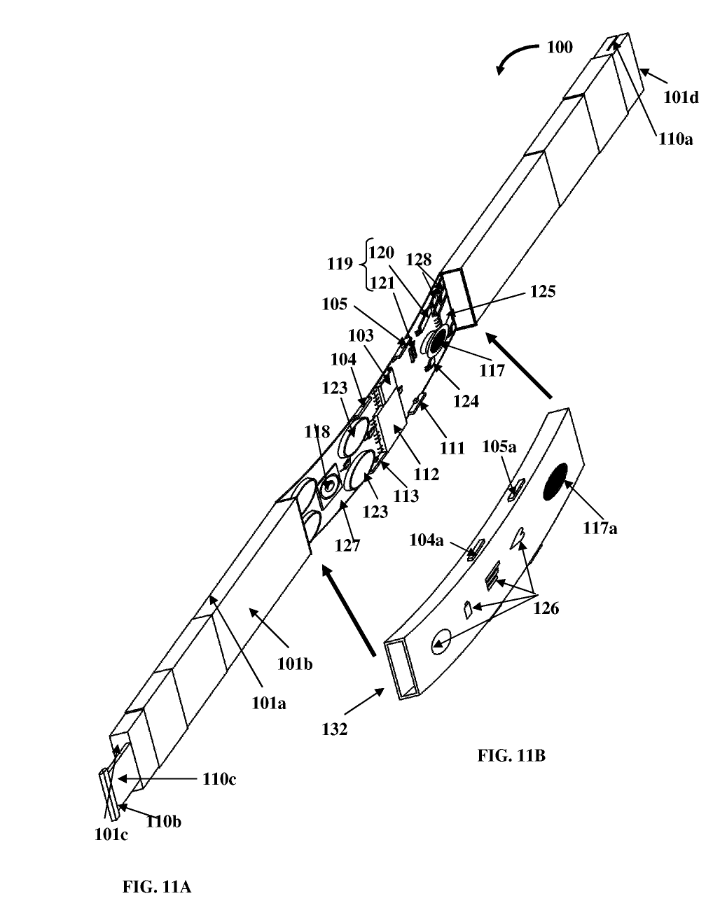

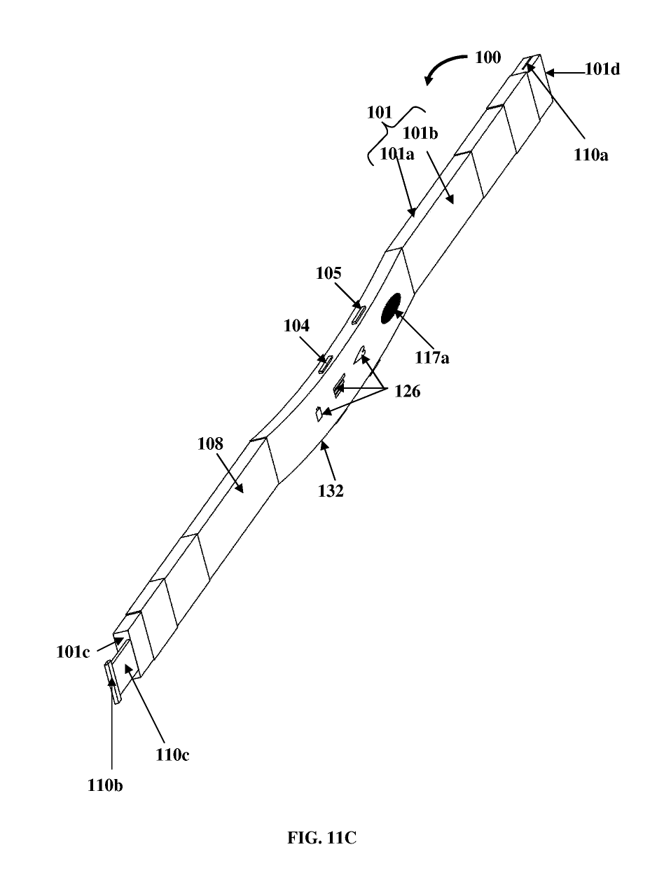

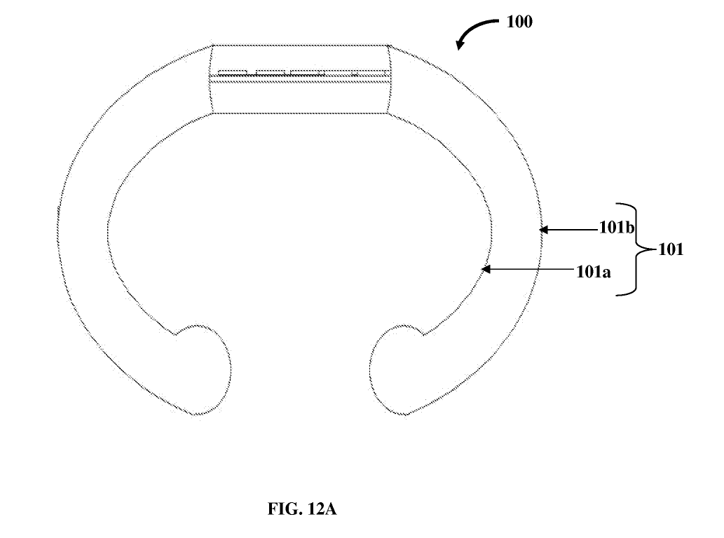

FIGS. 11A-11B exemplarily illustrate a perspective view of an embodiment of the portable monitoring device 100, showing a detachable section 132 that houses light emitting diodes 126 for indicating operating status of the portable monitoring device 100. The detachable section 132 is detached from the housing 101 of the portable monitoring device 100. The detachable section 132 of the portable monitoring device 100 houses a marker button socket 104a for connecting the marker button 104 and a function button socket 105a for connecting the function button 105 as disclosed in the detailed description of FIG. 7B. The detachable section 132 comprises an alerting unit housing 117a for enclosing the alerting unit 117. FIG. 11C exemplarily illustrates a perspective view of the portable monitoring device 100, showing the detachable section 132 of FIG. 11B attached to the housing 101 of the portable monitoring device 100.

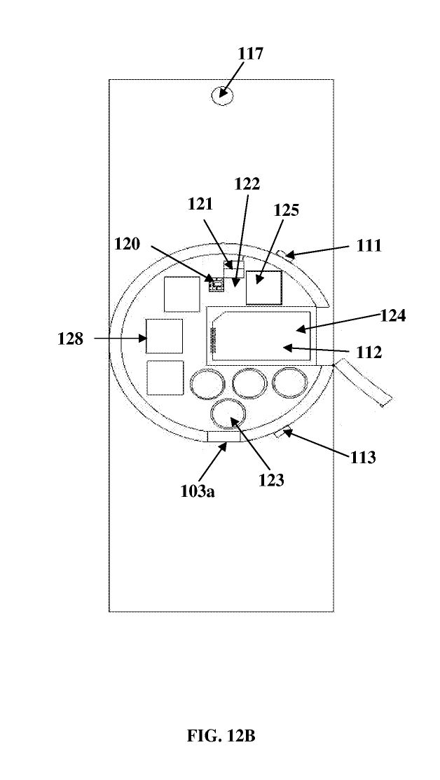

FIG. 12A exemplarily illustrates a side orthogonal view of an embodiment of the portable monitoring device 100 configured as an open-ended band. FIG. 12B exemplarily illustrates a top orthogonal view of an embodiment of the portable monitoring device 100 showing internal components 120, 121, 122, 123, 124, 125, 128, etc., of the portable monitoring device 100.

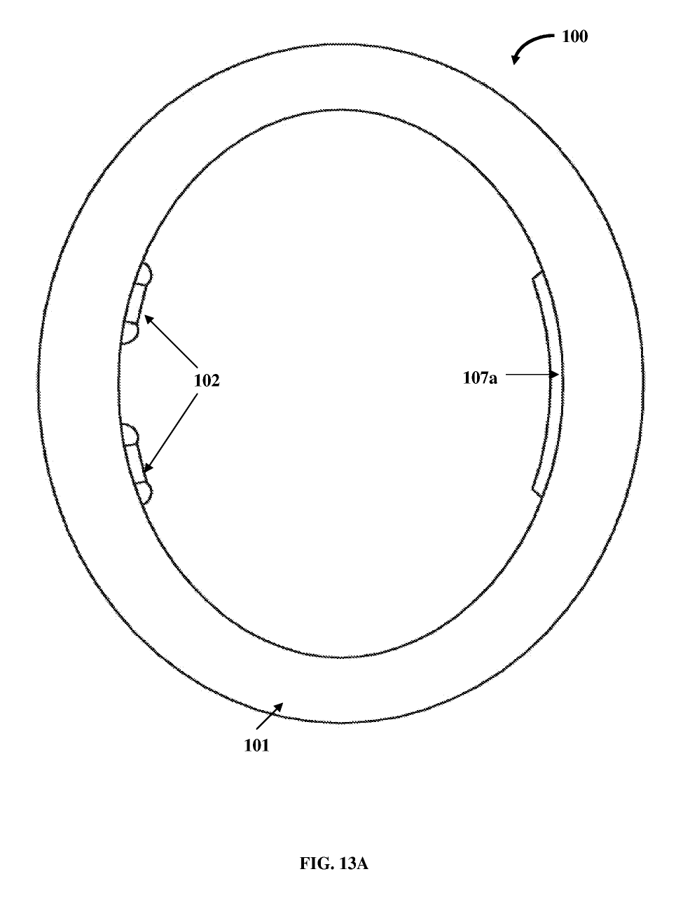

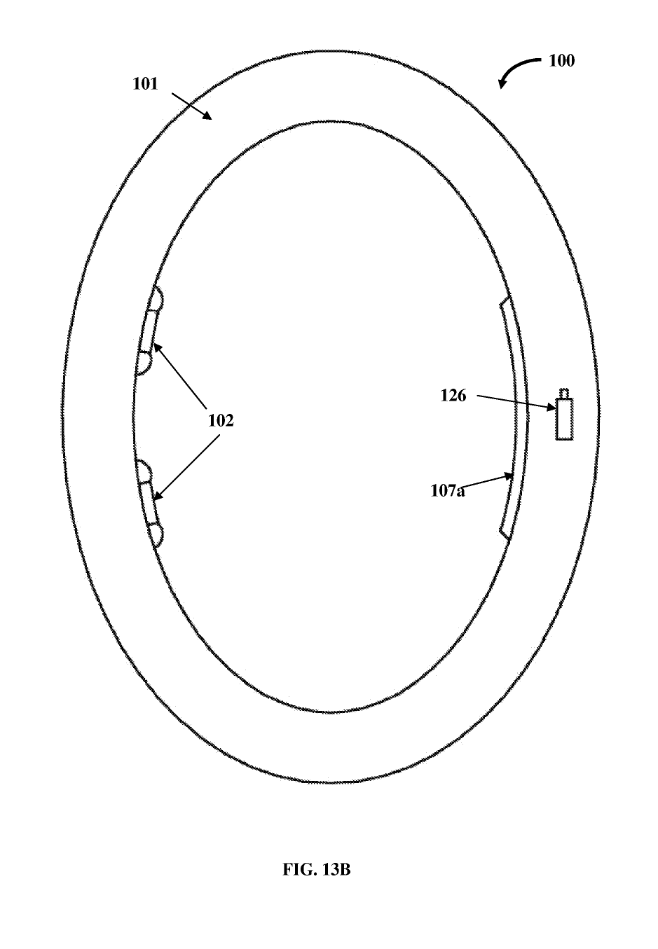

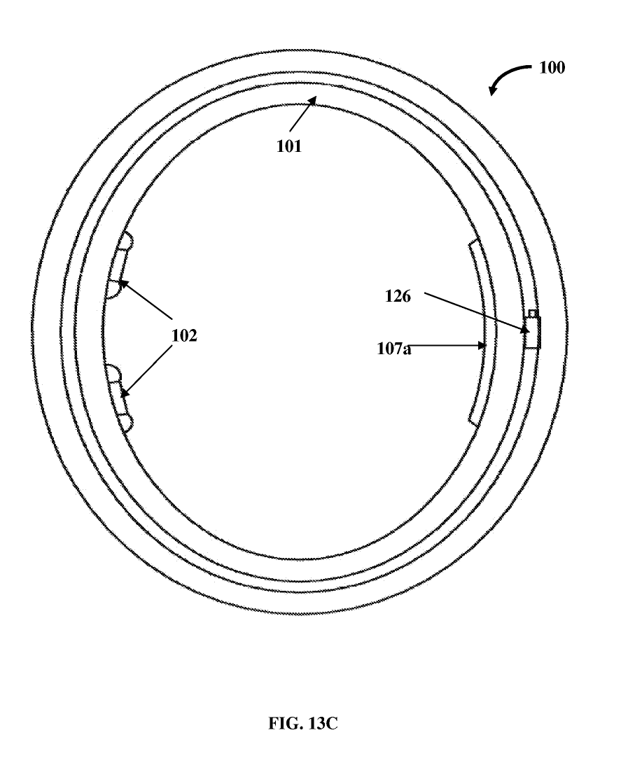

FIGS. 13A-13C exemplarily illustrate a portable monitoring device 100 configured to be placed on an infant's hand to monitor the infant at all times. The portable monitoring device 100 enables monitoring the condition of an infant all times. The parameter sensing devices 102 and 107a acquire physiological data associated with the physiological parameters, for example, the heartbeat, the temperature, etc., of the infant. The portable monitoring device 100 alerts a parent if and when, for example, the portable monitoring device 100 detects that the infant is suffering from abnormal breathing, irregular heart rate, elevated temperature, or another physiological condition monitored by the portable monitoring device 100.

FIGS. 14A-14B exemplarily illustrate orthogonal views showing a data communication unit 103 and a data communication interface 103a of the portable monitoring device 100. The data communication unit 103 is, for example, a secure digital removable flash memory device. The data communication unit 103 is accessible via the data communication interface 103a. The data communication unit 103 is connected within the housing 101 via the data communication interface 103a. The data communication unit 103 is capable of establishing communication with the base monitoring unit 1700 and the remote monitoring station 1803, for example, via a network 1802. The data communication unit 103 is capable of synchronizing with responder devices 1801. The data communication unit 103 transmits the processed physiological data and the patient information to the responder devices 1801 via the RF transceiver 124, the data communication interface 103a, and/or the RFID chip 122 for initiating relief measures. The data communication interface 103a establishes communication between one or more computer peripherals, for example, mice, keyboards, digital cameras, printers, personal media players, flash drives, network adapters, external hard drives, etc.

FIG. 15 exemplarily illustrates a portable monitoring device 100 configured as a sleeve garment 1501 for housing data communication interfaces 103a that interface peripheral devices 2000 to the portable monitoring device 100. FIG. 15 also illustrates an enlarged view of one of the data communication interfaces 103a on the sleeve garment 1501. The sleeve garment 1501 houses data communication interfaces 103a, for example, universal serial bus (USB) interfaces for interfacing with the peripheral devices 2000 as disclosed in the detailed description of FIG. 20. The sleeve garment 1501 is worn as a sleeve on the arm of the patient. The sleeve garment 1501 houses multiple data communication interfaces 103a to connect the peripheral devices 2000 to the portable monitoring device 100 to measure and acquire, for example, blood pressure, pulse oxygen, electrocardiography, etc. For example, a patient's blood pressure can be measured by using a blood pressure cuff that slides into a pocket inside the sleeve garment 1501. The portable monitoring device 100 drives the peripheral devices 2000. The data collected by the peripheral devices 2000 is relayed to the base monitoring unit 1700 and the remote monitoring station 1803 at regular intervals for storage. The sleeve garment 1501 is configured to provide comfort to the patient by avoiding multiple wires from being tangled.

FIG. 16 exemplarily illustrates an application of the portable monitoring device 100 for child care. Using the portable monitoring device 100, a parent can track a child wearing the portable monitoring device 100. The parent is notified by the portable monitoring device 100 if the child moves outside a predefined radius or if an emergency condition occurs with the child. The parent can communicate with the child via a two way communication user interface as disclosed in the detailed description of FIG. 4. In an embodiment, the Bluetooth chip 125 of the portable monitoring device 100 communicates with up to seven devices to form a wireless group, referred to as a piconet. The devices in the wireless group operate under a master-slave relationship, wherein the devices can switch roles by agreement for a slave device to become the master device, and vice versa. Data is transferred between the master device and the slave devices. The master device may switch rapidly from one device to another in a round robin fashion. Simultaneous transmission of data from the master device to multiple devices is possible in a broadcast mode. The master-slave relationship may also be enabled over cellular and GPS connections using cellular and GPS transceivers 1601. In an example, a parent may keep track of three kids in a theme park. The parent wearing the master portable monitoring device 100 can set geographical parameters, for example, a three mile radius on all slave portable monitoring devices 100 worn by the kids, and in the event that the slave portable monitoring devices 100 breach the parameters, the slave portable monitoring devices 100 send an alert to the master portable monitoring device 100 and appropriate action can be taken.

FIG. 17A exemplarily illustrate a front orthogonal view of a base monitoring unit 1700. The base monitoring unit 1700 is used for charging the portable monitoring device 100, storing the processed physiological data of the patient and the patient information received from the portable monitoring device 100, establishing communication with the remote monitoring station 1803 via one or more transceivers 1707 and data communication interfaces 1711a of the base monitoring unit 1700, and alerting the remote monitoring station 1803 during an emergency. The base monitoring unit 1700 comprises an image capture device 1701 for capturing images. The base monitoring unit 1700 further comprises user interface elements 1702, for example, for indicating the battery status on the portable monitoring device 100, for indicating the status of the image capture device 1701, for indicating the charge status of the individual portable monitoring devices 100, etc.

The base monitoring unit 1700 comprises a liquid crystal display 1703, device charging terminals 1704, an antenna 1706, a transceiver 1707, and a power supply 1708. The liquid crystal display 1703 provides a visual indication of, for example, the charge status of the individual portable monitoring devices 100. The base monitoring unit 1700 is powered, for example, by a direct current power supply 1708 for charging the portable monitoring devices 100. The device charging terminals 1704 are charged by the power supply 1708 through the wires 1704a. Multiple portable monitoring devices 100 can be connected to the device charging terminals 1704 of the base monitoring unit 1700 for charging their battery units 123. The base monitoring unit 1700 can charge multiple portable monitoring devices 100 at a time using the device charging terminals 1704. The base monitoring unit 1700 is configured to provide an emergency alert on the detection of abnormal vitals of the patient via the alerting unit 1705 over a network 1802. The base monitoring unit 1700 provides connectivity to the portable monitoring device 100 via a telephone jack 1712 as exemplarily illustrated in FIG. 7B, the data communication interface 1711a, and the antenna 1706. The base monitoring unit 1700 uploads information to the portable monitoring device 100 and downloads information from the portable monitoring device 100 using the transceiver 1707. The base monitoring unit 1700 enables software repairs and updates to be transmitted to the portable monitoring device 100.

FIG. 17B exemplarily illustrates a rear orthogonal view of the base monitoring unit 1700. The base monitoring unit 1700 further comprises a storage unit 1709, for example, a flash memory device for storing the physiological data of the patient. The base monitoring unit 1700 further comprises a modem 1710, one or more data communication units 1711, a socket 1712, a connection port 1713, one or more battery units 1714, a secondary storage unit 1715, a Wi-Fi card 1716, an antenna 1717, and a panic button 1718. The modem 1710 facilitates network connectivity with the remote monitoring station 1803 and the portable monitoring device 100. The data communication units 1711 enable transmission of data to the remote monitoring station 1803. The socket 1712, for example, a phone jack allows connection to a voice communication device. The connection port 1713, for example, an Ethernet port facilitates internet connectivity. The battery unit 1714 powers the base monitoring unit 1700. The secondary storage unit 1715, for example, a hard drive, a database on a hard drive, etc., stores physiological data of the patient and the patient information. The Wi-Fi card 1716 facilitates wireless connectivity. The antenna 1717 increases network coverage. The panic button 1718 can be activated for triggering a panic alert during an emergency situation.

FIGS. 17C-17D exemplarily illustrates a top orthogonal view and a bottom orthogonal view of the base monitoring unit 1700 respectively. The top orthogonal view exemplarily illustrated in FIG. 17C shows the device charging terminals 1704 and another alerting unit 1705. The base monitoring unit 1700 can charge multiple portable monitoring devices 100 at a time using the device charging terminals 1704. The alerting unit 1705 provide an emergency alert on the detection of abnormal vitals of the patient by the portable monitoring device 100. The bottom orthogonal view exemplarily illustrated in FIG. 17D shows the battery unit 1714 and the secondary storage unit 1715 of the base monitoring unit 1700 as disclosed in the detailed description of FIG. 17B.

FIG. 18 illustrates a system 1800 for monitoring and alerting physiological parameters of a patient using the portable monitoring device 100. The system 1800 disclosed herein comprises the portable monitoring device 100 as disclosed in the detailed description of FIGS. 1A-1B, FIGS. 2A-2D, FIGS. 3A-3B, and FIGS. 4-16, a base monitoring unit 1700 as disclosed in the detailed description of FIGS. 17A-17D, and a remote monitoring station 1803 communicating via a network 1802. The base monitoring unit 1700 communicates with the portable monitoring device 100 and the remote monitoring station 1803 over the network 1802. The base monitoring unit 1700 comprises one or more data communication units 1711 accessible via one or more data communication interfaces 1711a, a transceiver 1707, user interface elements 1702, device charging terminals 1704, and a storage unit 1715. The base monitoring unit 1700, in communication with the portable monitoring device 100, charges the portable monitoring device 100 via the device charging terminals 1704, stores the processed physiological data of the patient and the patient information received from the portable monitoring device 100 in the storage unit 1715, and establishes communication with the remote monitoring station 1803, for example, via one more RF transceivers 1707 and data communication interfaces 1711a. The data communication units 1711 transmit the processed physiological data of the patient and the patient information to the remote monitoring station 1803 via the network 1802. The remote monitoring station 1803 establishes communication with the portable monitoring device 100 for receiving the processed physiological data and the patient information from the portable monitoring device 100.

The system 1800 disclosed herein further comprises one or more responder devices 1801 capable of synchronizing with the data communication units 103 of the portable monitoring device 100 to retrieve the processed patient data and the patient information from the portable monitoring device 100. The responder devices 1801 are also capable of establishing communication with the remote monitoring station 1803 over the network 1802. The remote monitoring station 1803 may transmit the location information of the patient to the responder devices 1801 for initiating relief measures to the patient. The RF transceivers 124 and the data communication interfaces 103a of the portable monitoring device 100 communicate with the responder devices 1801, for example, using Bluetooth, radio frequency identification (RFID), universal serial bus (USB) communications, etc. The responder devices 1801 comprise a radio frequency identification (RFID) reader chip 1801a for scanning the portable monitoring device 100. The responders carrying the responder devices 1801 can connect the responder devices 1801 to the portable monitoring device 100 to synchronize data transfer between the responder devices 1801 and the portable monitoring device 100 to retrieve physiological data and the patient information, for example, allergies, medicines being taken, hospital preferences, emergency contacts, etc., of the patient, prior to initiating relief measures.

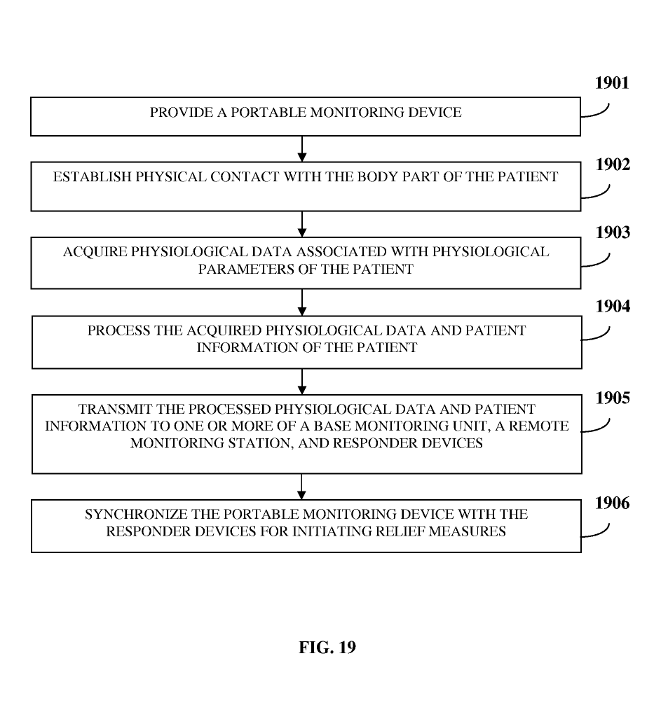

FIG. 19 illustrates a method for monitoring and alerting physiological parameters of a patient using the portable monitoring device 100. The portable monitoring device 100 as disclosed in the detailed description of FIGS. 1A-1B, FIGS. 2A-2D, FIGS. 3A-3B, and FIGS. 4-18 is provided 1901. The inner surface 101a of the housing 101 establishes 1902 physical contact with the patient's body part. The parameter sensing devices 102, 107a, 107b, etc., acquire 1903 physiological data associated with the physiological parameters of the patient as disclosed in the detailed description FIG. 5. The processing unit 127 processes 1904 the acquired physiological data and patient information of the patient. One or more data communication units 103 as disclosed in the detailed description FIGS. 14A-14B transmit 1905 the processed physiological data and the patient information to one or more of the base monitoring unit 1700, the remote monitoring station 1803, and one or more responder devices 1801 via one or more transceivers 124 and data communication interfaces 103a disposed within the housing 101 of the portable monitoring device 100 as disclosed in the detailed description of FIG. 18. The portable monitoring device 100 synchronizes 1906 with the responder devices 1801 for initiating relief measures. The portable monitoring device 100 therefore monitors the physiological parameters of the patient and alerts the remote monitoring station 1803 for providing the relief measures to the patient.

FIG. 20 exemplarily illustrates multiple peripheral devices 2000 capable of connecting to the portable monitoring device 100 via multiple data communication interfaces 103a for monitoring and alerting physiological parameters of a patient. The peripheral devices 2000 comprise, for example, an electrocardiography monitor 2001 connected to the portable monitoring device 100 via electrocardiography pads through a single data communication interface 103a. The peripheral devices 2000 further comprise, for example, a pulse oximeter 2002 located on either the fingertip or the earlobe of the patient for monitoring oxygen saturation of a patient's blood, a mouth piece 2003 connected to the portable monitoring device 100 for functioning as a breath analyzer for analyzing exhaled breath of a patient, an end-tidal carbon dioxide (EtCO2) monitor 2017 for measuring exhaled carbon dioxide of the patient, and an electroencephalography (EEG) peripheral 2004 connected to the portable monitoring device 100 for monitoring seizures associated with the patient's brain and alerting in the event of an emergency situation. The electroencephalography peripheral 2004 may also store information for use by a physician. The peripheral devices 2000 further comprise, for example, a bi-level positive airway pressure peripheral 2005 connected to the portable monitoring device 100, a handheld personal digital assistant device 2006 connected to the portable monitoring device 100 to synchronize the portable monitoring device 100 with emergency medical technicians, hospitals and physicians, and chargers or adapters 2007 connected to the portable monitoring device 100. The peripheral devices 2000 further comprise, for example, an emergency kit 2008, which is made available when a medical emergency occurs. The emergency kit 2008 attaches one or more peripheral devices 2000 to the portable monitoring device 100 via the data communication interfaces 103a.

The peripheral devices 2000 further comprise, for example, a video screen 2009, a speaker or headphone 2010, and a cell phone 2011 connected to the portable monitoring device 100. The peripheral devices 2000 further comprise, for example, a universal serial bus expansion port 2012 to allow multiple peripheral devices 2000 to be connected to the data communication interfaces 103a, a glucometer 2013 for monitoring the approximate quantity of glucose in the blood, a motion and movement monitor 2014 for monitoring the motion and movement of the patient wearing the portable monitoring device 100, and a pedometer 2015 for counting the steps taken by the patient.

The peripheral devices 2000 further comprise, for example, a nasal unit 2016 with a mouth piece 2003 for obstructed sleep apnea or any form of respiratory disease. The portable monitoring device 100 may be configured to have functionality that conforms to different international standards and is contemplated for use on a world wide scale, for example, in case of a traveler or individuals residing on all continents.

In another embodiment, the method and system 1800 disclosed herein comprises one or more peripheral devices 2000 connected to the portable monitoring device 100 via multiple data communication interfaces 103a for monitoring and alerting physiological parameters of the patient. The portable monitoring device 100 is designed to work in conjunction with a variety of medically implantable devices to monitor or support various physiological functions. These devices may include but are not limited to pace makers, automated implantable cardioverter-defibrillator (AICD), and intracranial devices.

Consider an example where a patient with a weak heart condition is prescribed with the portable monitoring device 100 by a medical health care professional to monitor and alert physiological parameters of the patient to the remote monitoring station 1803. The patient wears the portable monitoring device 100 on the wrist at all times. The physiological data associated with the physiological parameters of the patient is acquired on a continual basis by the parameter sensing devices 102, 107a, 107b, etc., of the portable monitoring device 100 via one or more touch points established by the contact of the inner surface 101a of the housing 101 with the wrist of the patient. The processing unit 127 processes the acquired physiological data of the patient. If the processed physiological data of the patient indicates undesired variations compared to the baseline values prescribed by the medical health care professional, the portable monitoring device 100 alerts the remote monitoring station 1803 of the condition of the patient. The portable monitoring device 100 transmits the patient information to the remote monitoring station 1803. Furthermore, the location determination unit 119 of the portable monitoring device 100 determines the location of the patient and transmits the location of the patient to the remote monitoring station 1803. The remote monitoring station 1803 transmits the location and the patient information to emergency responders carrying the responder devices 1801. The emergency responders arrive at the location of the patient and obtain the processed physiological data from the portable monitoring device 100 by synchronizing the responder devices 1801 with the portable monitoring device 100. The emergency responders can acquire the processed physiological data from the patient including current physiological real time data acquired from the portable monitoring device 100 prior to arriving. The emergency responders initiate relief measures for the patient based on the acquired physiological data.

The portable monitoring device 100 is designed to work seamlessly with proprietary software and systems. Using the data communication interface 103a, for example, the universal serial bus (USB) port and Wi-Fi capabilities, the satellite antenna/RF transceiver 124, a notification can be sent immediately when the physiological data exceeds or goes below the baseline levels. The portable monitoring device 100 automatically sends a message to the remote monitoring station 1803 that tries to reach the patient over the network 1802. If no response is received from the patient, the remote monitoring station 1803 notifies the emergency medical services personnel via the responder devices 1801 for initiating the relief measures.

The foregoing examples have been provided merely for the purpose of explanation and are in no way to be construed as limiting of the present invention disclosed herein. While the invention has been described with reference to various embodiments, it is understood that the words, which have been used herein, are words of description and illustration, rather than words of limitation. Further, although the invention has been described herein with reference to particular means, materials and embodiments, the invention is not intended to be limited to the particulars disclosed herein; rather, the invention extends to all functionally equivalent structures, methods and uses, such as are within the scope of the appended claims. Those skilled in the art, having the benefit of the teachings of this specification, may affect numerous modifications thereto and changes may be made without departing from the scope and spirit of the invention in its aspects.

We claim:

1. A portable monitoring device comprising:
a housing configured to conform to a body part of a patient, wherein said housing defines an inner surface for establishing physical contact with said body part of said patient, and an outer surface opposing said inner surface;
one or more parameter sensing devices located at predetermined positions on said inner surface of said housing for acquiring physiological data associated with physiological parameters of said patient by establishing said physical contact with said body part of said patient;
a processing unit disposed within said housing and in communication with said one or more parameter sensing devices for processing said acquired physiological data and patient information of said patient; and
one or more data communication units disposed within said housing and in communication with said processing unit for transmitting said processed physiological data and said patient information to one or more of a base monitoring unit, a remote monitoring station, and responder devices via one or more transceivers and data communication interfaces disposed within said housing, wherein one or more of said one or more data communication units are capable of synchronizing with said responder devices for initiating relief measures;
whereby said portable monitoring device monitors said physiological parameters of said patient and alerts said remote monitoring station for providing said relief measures to said patient.

2. The portable monitoring device of claim 1, further comprising a storage unit disposed within said housing for storing said acquired physiological data and said patient information and for providing backup of said acquired physiological data and said patient information.

3. The portable monitoring device of claim 1, further comprising one or more location determination units disposed within said housing and in communication with said processing unit for determining geographic location of said patient.

4. The portable monitoring device of claim 1, wherein said housing comprises a fastening and resizing assembly comprising one or more extensible sliding members for one of resizing said portable monitoring device to conform to said body part of said patient and repositioning said one or more parameter sensing devices on said housing to pick up optimum readings associated with said physiological parameters of said patient, wherein said fastening and resizing assembly further comprises a clasp for securing said portable monitoring device to said body part of said patient.

5. The portable monitoring device of claim 1, wherein said physiological parameters of said patient comprise one or more of heartbeat and electrocardiograph of said patient, temperature of said patient, and perspiration of said patient.

6. The portable monitoring device of claim 1, wherein said patient information comprises one of health information of said patient, billing information of said patient, insurance information of said patient, historical patient information, and any combination thereof.

7. The portable monitoring device of claim 1, further comprising one or more user interface elements located at predetermined positions on said housing for providing an audiovisual indication of one of functioning of said portable monitoring device, power levels of said portable monitoring device, disconnection of said one or more parameter sensing devices, and operating status of said portable monitoring device.

8. The portable monitoring device of claim 1, further comprising an alerting unit in communication with said processing unit for one of remotely alerting said remote monitoring station and providing local alerts via one of an audio mode, a visual mode, and any combination thereof, when said acquired physiological data associated with said physiological parameters of said patient exceeds baseline parameter values.

9. The portable monitoring device of claim 1, further comprising a unique identifier provided on one of said outer surface and said inner surface of said housing for one of identifying said patient associated with said portable monitoring device and logging said acquired physiological data and said patient information into a medical account of said patient for retrieval of said acquired physiological data and said patient information.

10. The portable monitoring device of claim 1, wherein said one or more transceivers transmit said processed physiological data and said patient information to said one or more of said base monitoring unit, said remote monitoring station, and said responder devices in one of a wired mode of communication, a wireless mode of communication, and a combination thereof.

11. The portable monitoring device of claim 1, further comprising emergency contact information provided on one of said inner surface and said outer surface of said housing of said portable monitoring device for enabling provision of said relief measures to said patient.

12. The portable monitoring device of claim 1, further comprising one or more battery units located inside said housing of said portable monitoring device for powering said portable monitoring device.

13. The portable monitoring device of claim 1, wherein said processed physiological data and said patient information transmitted to said remote monitoring station by said one or more transceivers are updated in a medical history log of said patient in said remote monitoring station.

14. A system for monitoring and alerting physiological parameters of a patient, comprising:
a portable monitoring device comprising:
a housing configured to conform to a body part of said patient, wherein said housing defines an inner surface for establishing physical contact with said body part of said patient, and an outer surface opposing said inner surface;
one or more parameter sensing devices located at predetermined positions on said housing for acquiring physiological data associated with said physiological parameters of said patient by establishing said physical contact with said body part of said patient;
a processing unit disposed within said housing and in communication with said one or more parameter sensing devices for processing said acquired physiological data and patient information of said patient; and
one or more data communication units disposed within said housing and in communication with said processing unit for transmitting said processed physiological data and said patient information to one or more of a base monitoring unit, a remote monitoring station, and responder devices via one or more transceivers and data communication interfaces disposed within said housing, wherein one or more of said one or more data communication units are capable of synchronizing with said responder devices for initiating relief measures;
said base monitoring unit in communication with said portable monitoring device for one of charging said portable monitoring device, storing said acquired physiological data of said patient and said patient information, and establishing communication with said remote monitoring station via one or more transceivers and data communication interfaces of said base monitoring unit;
said remote monitoring station in communication with said portable monitoring device, wherein said remote monitoring station receives said processed physiological data and said patient information from said portable monitoring device; and
one or more of said responder devices in communication with said remote monitoring station, wherein said responder devices synchronize with said one or more data communication units in said portable monitoring device via said one or more transceivers and said data communication interfaces of said portable monitoring device to retrieve said processed physiological data and said patient information from said portable monitoring device.

15. The system of claim 14, wherein said portable monitoring device is configured as a sleeve garment for housing a plurality of said data communication interfaces for connecting a plurality of peripheral devices to said portable monitoring device.

16. A method for monitoring and alerting physiological parameters of a patient, comprising:
providing a portable monitoring device comprising:
a housing configured to conform to a body part of said patient, wherein said housing defines an inner surface for establishing physical contact with said body part of said patient, and an outer surface opposing said inner surface;
one or more parameter sensing devices located at predetermined positions on said inner surface of said housing;
a processing unit disposed within said housing and in communication with said one or more parameter sensing devices; and
one or more data communication units disposed within said housing and in communication with said processing unit, wherein one or more of said one or more data communication units are capable of synchronizing with responder devices for initiating relief measures;
acquiring physiological data associated with said physiological parameters of said patient by said one or more parameter sensing devices by establishing said physical contact with said body part of said patient;
processing said acquired physiological data and patient information of said patient by said processing unit;
transmitting said processed physiological data and said patient information to one or more of a base monitoring unit, a remote monitoring station, and said responder devices via one or more transceivers and data communication interfaces disposed within said housing of said portable monitoring device; and
synchronizing said portable monitoring device with said responder devices for initiating relief measures.

17. The method of claim 16, further comprising storing said acquired physiological data and said patient information and providing backup of said acquired physiological data and said patient information by a storage unit disposed within said housing of said portable monitoring device.

18. The method of claim 16, further comprising determining geographic location of said patient by one or more location determination units disposed within said housing and in communication with said processing unit of said portable monitoring device.

19. The method of claim 16, further comprising providing a fastening and resizing assembly on said housing of said portable monitoring device, wherein said fastening and resizing assembly comprises one or more extensible sliding members for one of resizing said portable monitoring device to conform to said body part of said patient and for repositioning said one or more parameter sensing devices on said housing to pick up optimum readings associated with said physiological parameters of said patient, wherein said fastening and resizing assembly further comprises a clasp for securing said portable monitoring device to said body part of said patient.

20. The method of claim 16, further comprising providing one or more user interface elements located at predetermined positions on said housing of said portable monitoring device for providing an audiovisual indication of one of functioning of said portable monitoring device, power levels of said portable monitoring device, disconnection of said one or more parameter sensing devices, and operating status of said portable monitoring device.

21. The method of claim 16, further comprising providing an alerting unit that communicates with said processing unit of said portable monitoring device for one of remotely alerting said remote monitoring station and providing local alerts via one of an audio mode, a visual mode, and any combination thereof, when said acquired physiological data associated with said physiological parameters of said patient exceeds baseline parameter values.

22. The method of claim 16, further comprising providing one or more battery units inside said housing of said portable monitoring device for powering said portable monitoring device.

23. The method of claim 16, further comprising providing a unique identifier on one of said inner surface and said outer surface of said housing of said portable monitoring device for one of identifying said patient associated with said portable monitoring device and logging said acquired physiological data and said patient information into a medical account of said patient for retrieval of said acquired physiological data and said patient information.

24. The method of claim 16, further comprising providing emergency contact information on one of said inner surface and said outer surface of said housing of said portable monitoring device for enabling provision of said relief measures to said patient.

25. The method of claim 16, further comprising providing said base monitoring unit that communicates with said portable monitoring device for one of charging said portable monitoring device, storing said physiological data of said patient and said patient information, and establishing communication with said remote monitoring station via one or more of said one or more transceivers and said data communication interfaces of said portable monitoring device.
